(19) United States
(12) Patent Application Publication
(10) Pub. No.: US 2016/0168138 A1
(43) Pub. Date: Jun. 16, 2016

(54) PROCESS FOR THE PREPARATION OF SUVOREXANT AND INTERMEDIATES USEFUL IN THE SYNTHESIS OF SUVOREXANT

(71) Applicant: DR. REDDY'S LABORATORIES LIMTED, Hyderabad (IN)

(72) Inventors: Javed IQBAL, Hyderabad (IN); Vilas Hareshwar DAHANUKAR, Hyderabad (IN); Srinivas ORUGANTI, Hyderabad (IN); Bhaskar KANDAGATLA, Hyderabad (IN)

(21) Appl. No.: 14/905,748

(22) PCT Filed: Jul. 15, 2014
(86) PCT No.: PCT/IB2014/063115

§ 371 Date: Jan. 15, 2016

(30) Foreign Application Priority Data
Jul. 15, 2013 (IN) 3165/CHE/2013

(51) Int. Cl. C07D 413/12; C07D 263/58

(52) U.S. Cl. CPC C07D 413/12; C07D 263/58

(57) ABSTRACT

A novel processes for the preparation of suvorexant (formula I), its related compounds and its intermediates that are simple, economical and commercially viable. (I)

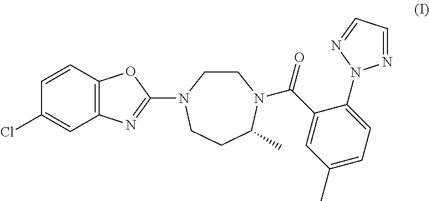 [SMILES: Cc1ccc(-n2nccn2)c(C(=O)N2CCN(c3nc4cc(Cl)ccc4o3)CC[C@H]2C)c1]

### FIELD OF THE INVENTION

The present invention provides novel synthetic processes for obtaining suvorexant, its related compounds and its intermediates.

### BACKGROUND

Suvorexant chemically described as [(R)-4-(5-Chlorobenzoxazol-2-yl)-7-methyl-[1,4]diazepan-1-yl]-(5-methyl-2-[1,2,3]triazol-2-yl-phenyl)methanone is an antagonist of orexin receptor. It can be structurally represented by the following formula I:

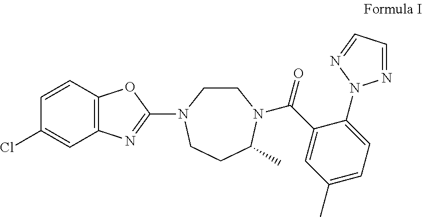 [SMILES: Cc1ccc(-n2nccn2)c(C(=O)N2CCN(c3nc4cc(Cl)ccc4o3)CC[C@H]2C)c1]

U.S. Pat. No. 7,951,797 discloses suvorexant and process for the preparation wherein 5-methyl-2-(1,2,3-triazol-2-yl) benzoic acid is coupled with 1-benzyloxycarbonyl-5(R)-methyl-1,4-diazepane hydrochloride using EDC, HOAt and NMM to produce benzyl(5R)-5-methyl-4-[5-methyl-2-(2H-1,2,3-triazol-2-yl)benzoyl]-1,4-diazepane-1-carboxylate, which is N-deprotected using H₂ over Pd(OH)₂/C to obtain (7R)-7-methyl-1-[5-methyl-2-(2H-1,2,3-triazol-2-yl)benzoyl]-1,4-diazepane[amine]. Subsequently amine is condensed with 2,5-dichlorobenzoxazole in the presence of triethyl amine to obtain suvorexant (Formula I).

WO2012148533 and Org. Process Res. Dev. 2011, 15, 367-375 (OPRD) discloses 5-Chloro-2-(5-methyl-[1,4]diazepan-1-yl)-benzoxazole(diazepine intermediate) as an intermediate for the synthesis of suvorexant. The preparation of diazepine intermediate is carried out by racemic direct reductive amination of 4-[(2-Amino-ethyl)-(5-chlorobenzoxazol-2-yl)amino]butan-2-one-bis-methane sulfonic acid salt with a reducing agent in the presence of a weak base, followed by chiral resolution. According to OPRD reference, weak base is added to prevent cleavage of benzoxazole moiety under the conditions of reductive amination. (R)-5-Chloro-2-(5-methyl-[1,4] diazepan-1-yl)-benzoxazole is reacted with 5-methyl-2-(1,2,3-triazol-2-yl)benzoic acid after its conversion into corresponding acid chloride, in presence of base to obtain suvorexant.

Org. Lett., Vol. 14, No. 13, 2012 discloses an asymmetric transamination of 4-[(2-Amino-ethyl)-(5-chlorobenzoxazol-2-yl)amino]butan-2-one-bis-methane sulfonic acid salt by biocatalytic transamination technology.

There remains a need to provide a novel processes for the preparation of suvorexant (formula I), its related compounds and its intermediates that are simple, economical and commercially viable.

### SUMMARY

The present invention provides novel synthetic processes for obtaining suvorexant (Formula I), its related compounds and its intermediates.

In a first embodiment, the present invention provides a process for preparing suvorexant (Formula I) comprising one or more steps (a) to (h) according to synthetic scheme I:

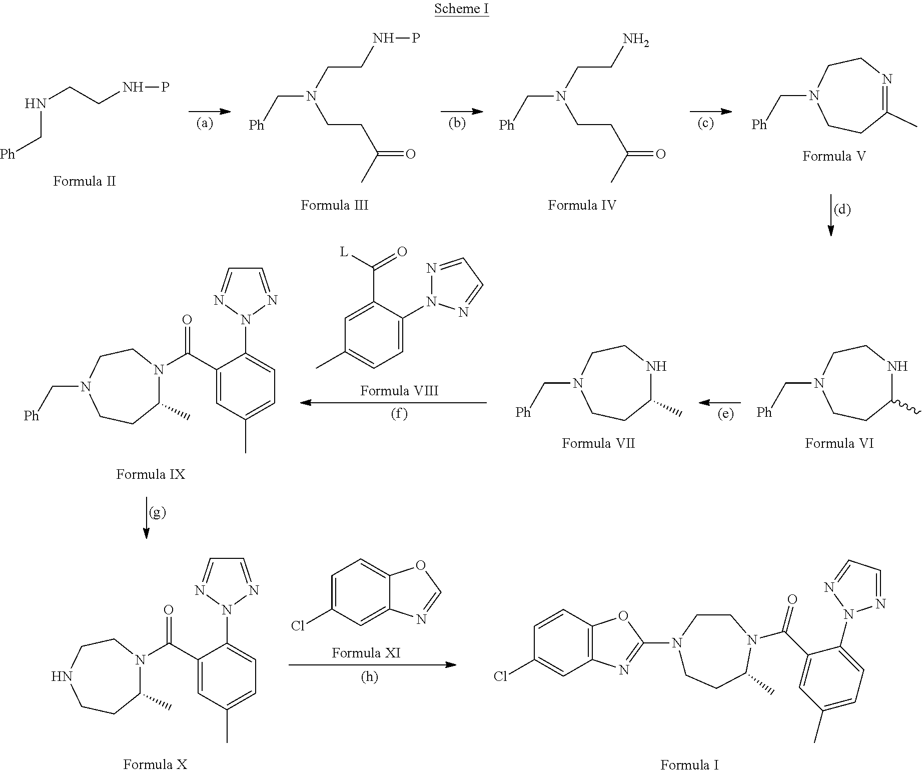

wherein P is an amino protecting group and L is a leaving group.

In a second embodiment, the present invention provides a process for preparing suvorexant (Formula I) comprising conversion of the compound of Formula II to the compound of formula III:

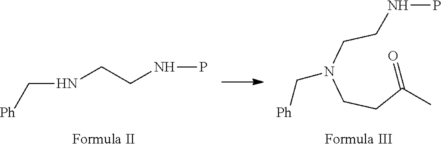

wherein P is an amino protecting group.

In a third embodiment, the present invention provides a process for preparing suvorexant (Formula I) comprising conversion of the compound of Formula III to the compound of formula IV:

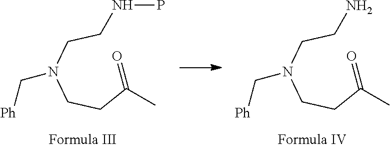

wherein P is an amino protecting group.

In a fourth embodiment, the present invention provides a process for preparing suvorexant (Formula I) comprising conversion of the compound of Formula IV to the compound of formula VI:

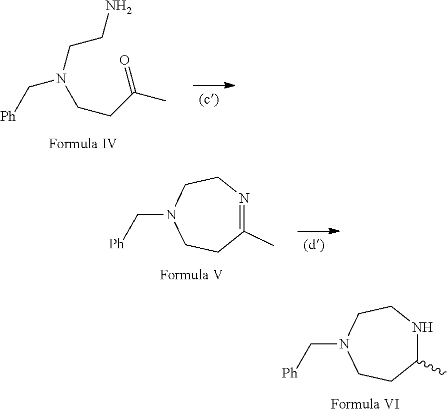 [SMILES: CC(=O)CCN(CCN)Cc1ccccc1 ; CC1=NCCN(Cc2ccccc2)CC1 ; CC1CCN(Cc2ccccc2)CCN1]

In a fifth embodiment, the present invention provides a process for preparing suvorexant (Formula I) comprising conversion of the racemic compound of Formula VI to the compound of formula VII that is enantiomerically enriched:

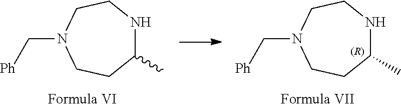

In a sixth embodiment, the present invention provides a process for preparing enantiomer of suvorexant (Formula Ia) comprising conversion of the racemic compound of Formula VI to the compound of formula VIIa that is enantiomerically enriched:

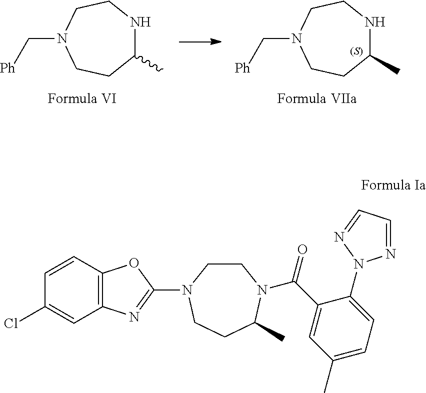

In a seventh embodiment, the present invention provides a process for preparing suvorexant (Formula I) comprising reaction of the enantiomerically enriched compound of Formula VII with the compound of formula VIII to provide the compound of formula IX:

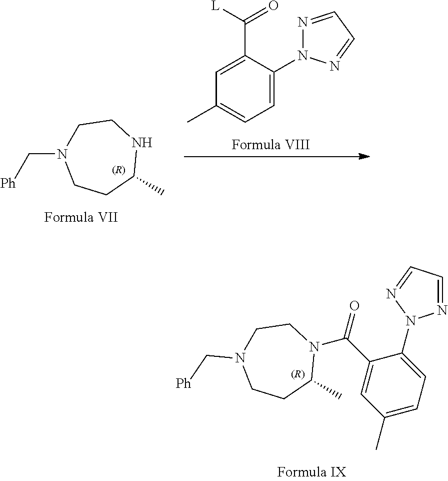

In an eighth embodiment, the present invention provides a process for preparing enantiomer of suvorexant (Formula Ia) comprising reaction of the enantiomerically enriched compound of Formula VIIa with the compound of formula VIII to provide the compound of formula IXa:

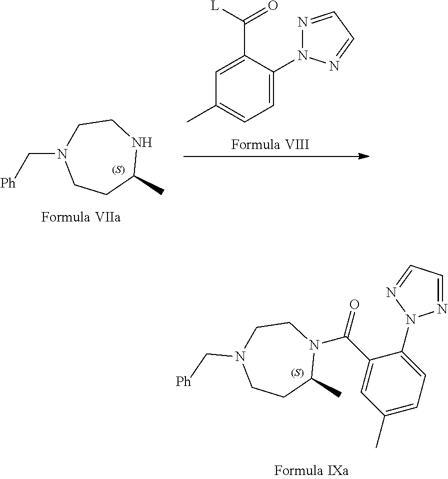

In a ninth embodiment, the present invention provides a process for preparing suvorexant (Formula I) comprising conversion of the compound of Formula IX to the compound of formula X:

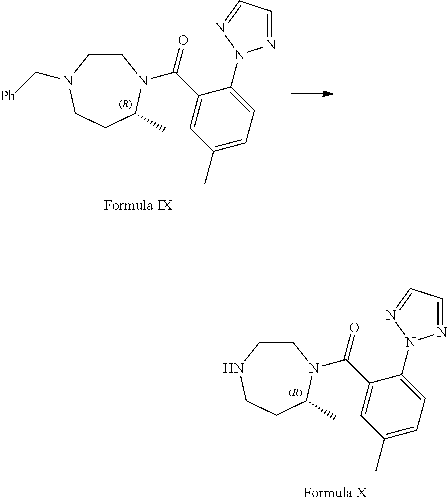

In a tenth embodiment, the present invention provides a process for preparing enantiomer of suvorexant (Formula Ia) comprising conversion of the compound of Formula IXa to the compound of formula Xa:

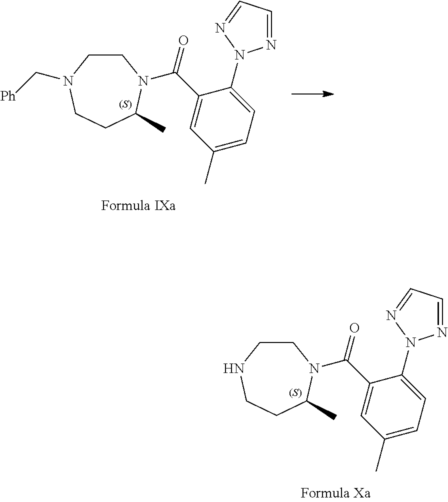

In an eleventh embodiment, the present invention provides a process for preparing suvorexant (Formula I) comprising reaction of the compound of Formula X with the compound of formula XI:

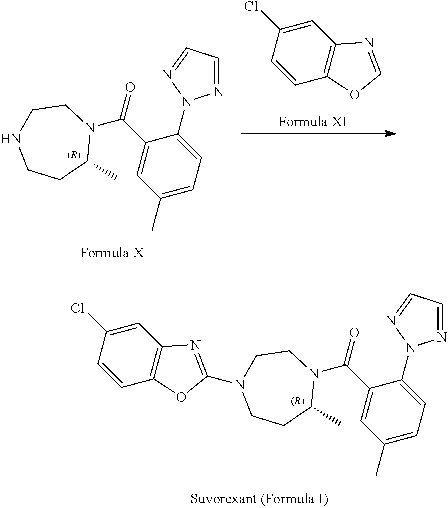

In a twelfth embodiment, the present invention provides a process for preparing enantiomer of suvorexant (Formula Ia) comprising reaction of a compound of Formula Xa with a compound of formula XIa:

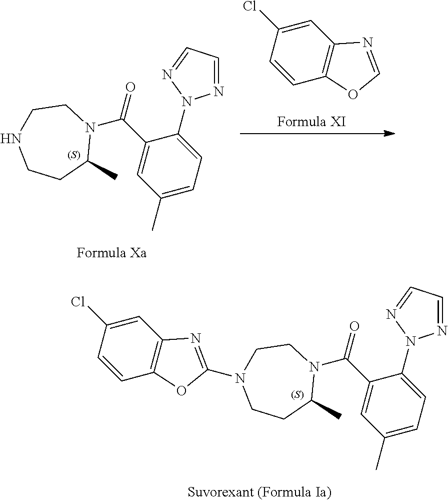

In a thirteenth embodiment, the present invention provides a process for preparing the compound of formula XXI:

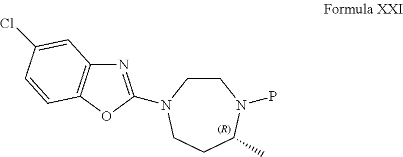

comprising:
(a) protecting the compound of formula VII to provide the compound of formula XXII:

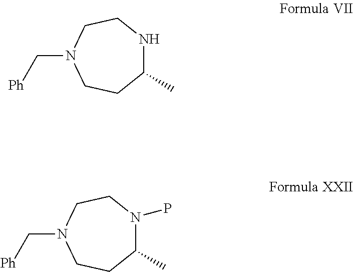

(b) converting the compound of formula XXII to obtain the compound of formula XXIII;

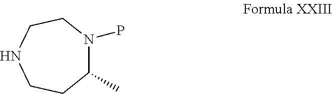

(c) reacting the compound of formula XXIII with the compound of formula XI:

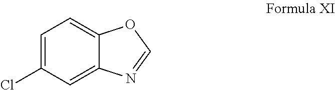

to provide a compound formula XXI.

In a fourteenth embodiment, the present invention provides a process for the preparation of a compound of formula XXV, which comprises:

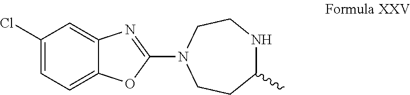 [SMILES: CC1CCN(c2nc3cc(Cl)ccc3o2)CCN1]

converting the compound of formula XXIV to the compound of formula XXV

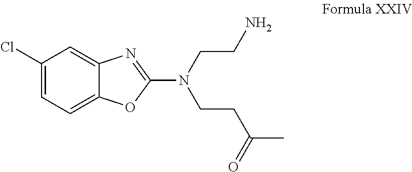 [SMILES: CC(=O)CCN(CCN)c1nc2cc(Cl)ccc2o1]

in presence of a reagent and a reducing agent.

The present invention further encompasses the steps (a) to (h) of scheme-1 depicted above independently.

### DETAILED DESCRIPTION

The term “about” when used in the present invention preceding a number and referring to it, is meant to designate any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

Optionally, in carrying out the processes according to the present invention, the reaction product of a given step can be carried forward to the next step without the isolation of the product i.e., one or more reactions in a given process can be carried out in-situ as one pot process optionally in the presence of the same reagent/s used in a previous step wherever appropriate to do so, to make the process of the present invention economical and commercially more viable.

Optionally, in carrying out the processes according to the present invention, the reaction product of a given step can be isolated and purified by the methods described herein or the methods known to a person skilled in the art before using in a subsequent step of the process.

In the present invention, the isolation of products after completion of the reactions can be effected by removing the solvent. Suitable techniques which can be used for the removal of the solvent include evaporation techniques such as a Büchi® Rotavapor®, spray drying, thin film drying, nauta drying, tray drying, freeze drying (lyophilization) or any other suitable technique.

Isolated product can be optionally further dried. Drying can be suitably carried out in a tray dryer, vacuum oven, Büchi® Rotavapor®, air oven, fluidized bed dryer, spin flash dryer, flash dryer, cone dryer, agitated nutsche filter cum dryer, nauta dryer or the like or any other suitable dryer. The drying can be carried out at atmospheric pressure or under reduced pressures at temperatures of less than about 150° C., less than about 100° C., less than about 60° C., less than about 40° C., less than about 20° C., less than about 0° C., less than about −20° C., or any other suitable temperatures. The drying can be carried out for any time period required for obtaining a desired quality, such as from about 15 minutes to several hours.

The dried product can be optionally milled to get desired particle sizes. Milling or micronization may be performed before drying, or after the completion of drying of the product. Techniques that may be used for particle size reduction include, without limitation, ball, roller, hammer mills and jet mills.

In an aspect, suvorexant may have a particle sizes of less than about 200 μm, less than about 150 μm, less than about 100 μm, less than about 90 μm, less than about 80 μm, less than about 60 μm, less than about 50 μm, less than about 40 μm, less than about 30 μm, less than about 20 μm, less than about 10 μm, less than about 5 μm or any other suitable particle sizes.

Particle size distributions of suvorexant particles may be measured using any techniques known in the art. For example, particle size distributions of suvorexant particles may be measured using microscopy or light scattering equipment, such as, for example, a Malvern Master Size 2000 from Malvern Instruments Limited, Malvern, Worcestershire, United Kingdom.

In different embodiments of the present invention, P in compound II and compound III represents an amino-protecting group. The term “N-protecting group” or “amino-protecting group” as used herein refers to those groups intended to protect a nitrogen atom against undesirable reactions during synthetic procedures. N-protecting group includes, aryloxycarbonyl such as benzyloxycarbonyl (Cbz), fluorenylmethoxycarbonyl (Fmoc); alkoxycarbonyl such as methyloxycarbonyl, acetoxycarbonyl, propoxycarbonyl, tert-butyloxycarbonyl (Boc); acyl such as acetyl, propanoyl, iso-butyryl, tert-butyryl, t-butylacetyl, pivaloyl; aroyl groups such as benzoyl; silyl such as trimethylsilyl, ter-butyldimethylsilyl; sulphonyl such as methanesulphonyl, p-tolylsulphonyl; sulphenyl such as 2-nitorphenylsulfenyl; urea; urethane; nitroso; nitro and the like. A thorough discussion of amino-protecting groups disclosed in Protective Groups in Organic Synthesis, Fourth edition, Wiley, New York 2006 by T. W. Greene and P. G. M. Wuts, which is incorporated herein by reference.

The compound of formula II, which is represented by the formula of —NHP, wherein P represents amino protecting group as described above. The compound of formula II can be prepared using amino protecting reagent. The term “amino-protecting reagent” as used herein refers to a compound that reacts with the amino functionality to give a protected amino group. For example, tert-butyloxycarbonyl protection can be prepared by a process reported in Bioorganic & Medicinal Chemistry, 18(3), 1135-1142, 2010. Similarly other amino protecting group can incorporated using amino-protecting reagents such as acylating reagents, sulfonylating reagents, sulfenylating reagents, urea and urethane-type reagents, nitroso derivatives, nitro derivatives, or silyl reagents. Various amino-protecting reagents have been described by Greene & Wuts in Protective Groups in Organic Synthesis. Person skilled in the art can choose individual reagent or reagent combinations based on desired protecting group. The reaction conditions for incorporation of protecting group can be optimized depending upon factors as the solubility of reagents, reactivity of reagents, preferred temperature ranges and suitable conditions for removing excess protecting reagent.

The amount of the amine-protecting reagent can vary depending on which amine-protecting reagent is used. Typically, the reaction can be accomplished with from about 1.0 to about 4.0 molar equivalents of the amino-protecting reagent relative to one molar equivalent of unprotected amine. Preferably, about 1.0 to about 1.5 molar equivalents of the amino-protecting reagent can be used. The reaction can be accomplished in the presence of an organic or inorganic base.

In different embodiments of the present invention, L in compound VII represents a leaving group which includes, halo (Cl, Br, I); hydroxy; alkoxy; aryloxy; alkanoate; aryloate; alkyl sulphonate; arylsulphonate; a substituted or unsubstituted or cyclic or acyclic amino that can form amide bond.

In the present invention, the compound of formula II can be reacted with the compound of formula XII to provide compound of Formula III.

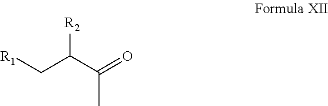

wherein R₁ includes halo (Cl, Br, I), alkyl sulphonyl, aryl sulphonyl, hydroxyl and R₂ is hydrogen; or R₁ and R₂ together form a bond; or an equivalent compound thereof. Preferably, compound of Formula XII includes, 4-halobutan-2-one, 3-oxobutyl methanesulfonate, 3-oxobutyl p-tolylsulfonate, 3-oxobutyl p-nitrobenzenesulfonates, pent-4-en-2-one or 4-hydroxybutan-2-one. More preferably, compound of formula XII can be pent-4-en-2-one.

In the present invention, the compound of formula II can be reacted with the compound of formula XII in presence of base. The base that can be used for the said reaction includes, organic base such as DBU (1,8-diazabicyclo[5.4.0]undec-7-ene), DBN (1,5-Diazabicyclo[4.3.0]non-5-ene), DABCO (1,4-diaza-bicyclo[2.2.2]octane), ABCO (1-azabicyclo[2,2,2]octane), TBD (1,5,7-Triazabicyclo[4.4.0]dec-5-ene) or DMAP (4-dimethylaminopyridine), TEA (Triethylamine), DIPEA (N,N-diisopropylethylamine), DIEA (Diethylamine), N-methyl morpholine, lutidine, pyridine or collidine; hydroxides of alkali metals such as sodium hydroxide, lithium hydroxide or potassium hydroxide; carbonates of alkali metals such as sodium carbonate or potassium carbonate; bicarbonates of alkali metals such as sodium bicarbonate or potassium bicarbonate.

Optionally, the reaction of the compound of formula II with a compound of formula XII can be carried out in the presence or absence of a solvent. The solvent that can be used in the said reaction includes, water; C₁-C₁₀ straight or branched chain alcohol such as methanol, ethanol, isopropyl alcohol, 1-butanol, 2-butanol, 2-methyl-2-propanol, 1-pentanol, 2-pentanol, 2,2-dimethyl-1-propanol, 2,2,2-trimethyl ethanol, 1-decanol; ethers such as tetrahydrofuran, 1,4-dioxane, diisopropylether, diethylether, 2-methyltetrahydrofuran, cyclopentyl methyl ether or methyl tert-butyl ether; esters such as ethyl acetate or isopropyl acetate; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, o-xylene, m-xylene or p-xylene; dimethyl sulphoxide, amide such as N,N-dimethyl formamide, N,N-dimethyl acetamide or mixtures thereof.

In the present invention, conversion of the compound of formula III to the compound of formula IV can be effected by selecting appropriate method known to persons skilled in the art. Based on the sensitivity of protecting group to pH, the pH of the reaction mixture can be adjusted for removal of protecting group. Various methods for deprotection of an amino protecting have been described in Chem. Rev. 2009, 2455-2504; which is incorporated herein by reference. For example, when protecting group is alkoxycarbonyl, deprotection can be carried out using acid, lewis acid or water. The acid that can be used includes trifluoacetic acid, hydrochloric acid, p-toluenesulfonic acid, methanesulfonic acid, trifluoromethanesulfonic acid or aqueous phosphoric acid. The lewis acid that can be used includes, BF₃.OEt₂, TMSI, TMSOTf, TiCl₄, SnCl₄, AlCl₃, Sn(OTf)₂, ZnBr₂, FeCl₃, InBr₃, Sc(OTf)₃, InCl₃, Yb(OTf)₃, or ZnCl₂.

The conversion of the compound of formula IV to the compound of formula V or the conversion of the compound of formula IVa to the compound of formula Va can be carried out using a suitable reagent. The said suitable reagent can be an acid or a reagent capable of releasing an acid in situ. The suitable acid includes, methanesulfonic acid, ethanesulfonic acid, benzenesulfonic acid, p-toluenesulfonic acid, nitric acid, sulfuric acid, phosphoric acid, hydrochloric acid, hydrobromic acid, tartaric acid, citric acid, acetic acid, and maleic acid. The reagent capable of releasing an acid in situ includes, but are not limited to, cyanuric halide (Formula XII), trihaloisocyanuric acid (Formula XIV), N-halosuccinimide (Formula XV), Tetrahaloglycouril (Formula XVI), 1,3-dihalo-5,5-dimethyl-hydantoin (Formula XVII), 1,3-dihalo-5,5-diphenyl-hydantoin (Formula XVIII), N-halophthalimide (Formula XIX), or N-haloacetamide (Formula XX).

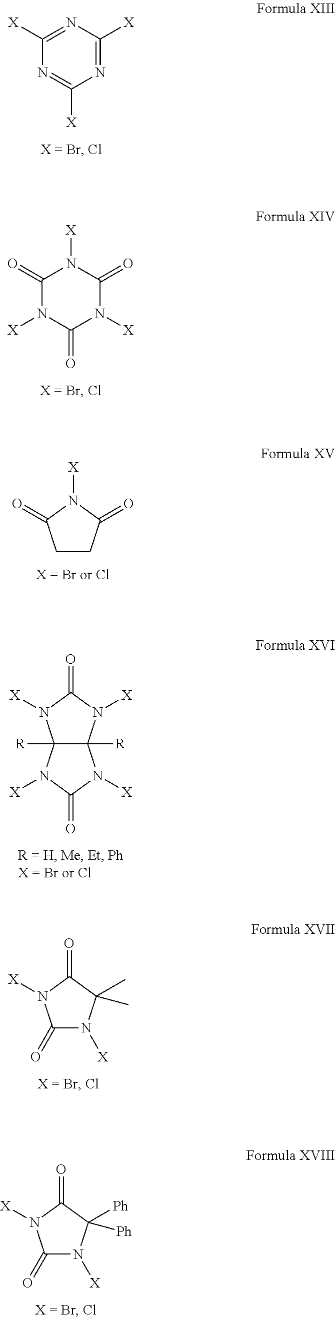

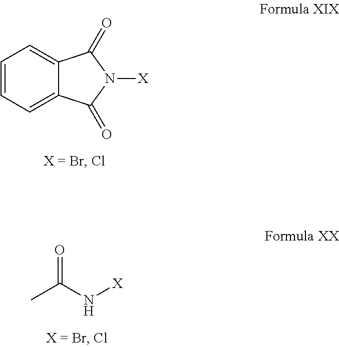

The molar ratio of the acid or the reagent capable of releasing an acid in situ with respect to the compound of Formula IV or the compound of formula IVa can be easily derived by a person skilled in the art. For example, the said mole ratio can be about 0.01, about 0.02, about 0.05, about 0.1, about 0.2, about 0.5, about 1.0, about 1.5, or about 2 mole per mole of the compound of formula IV, or any other suitable mole ratio.

The conversion of the compound of formula IV to the compound of formula V or the conversion of the compound of formula IVa to the compound of formula Va can be carried out in the presence or absence of a solvent. The suitable solvent includes, C₁-C₁₀ straight or branched chain alcohol such as methanol, ethanol, isopropyl alcohol, 1-butanol, 2-butanol, 2-methyl-2-propanol, 1-pentanol, 2-pentanol, 2,2-dimethyl-1-propanol, 2,2,2-trimethyl ethanol, 1-decanol, benzyl alcohol; ethers such as tetrahydrofuran, dioxane, diisopropylether, diethylether, 2-methyltetrahydrofuran, cyclopentyl methyl ether or methyl tert-butyl ether; esters such as ethyl acetate, isopropyl acetate; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, chlorobenzene, 4-chlorotoluene, trifluorotoluene, o-xylene, m-xylene or p-xylene, or mixtures thereof.

The conversion of the compound of formula IV to the compound of formula V or the conversion of the compound of formula IVa to the compound of formula Va can take place at a temperature of about −20° C. to about 150° C., about 10° C. to about 100° C., about 20° C. to about 50° C., about room temperature, about reflux temperature of the solvent used in the reaction, or any other suitable temperature, which facilitates the desired reaction to happen without substantially negatively affecting the quality of the substrates or the reaction product.

Room temperature as used herein refers to ‘the temperatures of the thing close to or same as that of the space, e.g., the room or fume hood, in which the thing is located’. Typically, room temperature can be from about 20° C. to about 30° C., about 22° C. to about 27° C., or about 25° C.

The reaction time should be sufficient to complete the reaction which depends on scale and mixing procedures, as is commonly known to one skilled in the art. Typically, the reaction time can vary from about few minutes to several hours. For example the reaction time can be from about 10 minutes to about 24 hours, or any other suitable time period.

In the present invention, a compound of formula V can be reduced using reducing agent to obtain the compound of formula VI. The suitable reducing agent includes, borohydrides such as sodium borohydride, potassium borohydride, lithium borohydride, sodium cyanoborohydride, potassium cyanoborohydride, lithium cyanoborohydride, sodium triacetoxyborohydride, potassium triacetoxyborohydride, also in the presence of suitable additives such as sulfuric acid, methanesulfonic acid, acetic acid, titanium chloride, zinc chloride, cobalt (II) chloride, aluminium chloride, tin chloride, nickel chloride, phosphorus oxychloride, methanesulfonic anhydride, trifluoromethanesulfonic anhydride, pyridine, iodine, trifluoroethanol or 1,2-ethanedithiol; boranes such as borane, diborane or catechol borane, also in the form of complexes with ethers, sulfides or amines such as BH₃SMe₂, BH₃.Et₂O, BH₃.THF, BH₃-t-butylamine, BH₃-dimethylamine or BH₃.diethylaniline; silanes such as triethylsilane, diphenylsilane or trichlorosilane, optionally in the presence of one or more Lewis acids, such as trifluoroborane, titanium chloride, aluminium chloride, zinc iodide or trifluoroacetic acid, also in form of complexes with ethers, such as boron trifluoride diethyl etherate; aluminium hydrides such as aluminium hydride (alane), LiAlH₄, ⁱBu₂AlH, sodium bis(2-methoxyethoxy)aluminium hydride (Red-Al) or LiHAl(OCH₃)₂, optionally in the presence of one or more Lewis acids, such as trifluoroborane, titanium chloride, aluminium chloride, zinc iodide or trifluoroacetic acid.

The molar ratio of the reducing agent that can be used with respect to the compound of formula IV can be easily derived by a person skilled in the art. For example, the said mole ratio can be about 0.01, about 0.02, about 0.05, about 0.1, about 0.2, about 0.5, about 1.0, about 1.5, about 2 or any other suitable mole per mole of the compound of formula IV.

The reduction of the compound of formula V to provide the compound of formula VI can be carried out in the presence or absence of a solvent. The suitable solvent includes, C₁-C₁₀ straight or branched chain alcohol such as methanol, ethanol, isopropyl alcohol, 1-butanol, 2-butanol, 2-methyl-2-propanol, 1-pentanol, 2-pentanol, 2,2-dimethyl-1-propanol, 2,2,2-trimethyl ethanol, 1-decanol, benzyl alcohol; ethers such as tetrahydrofuran, dioxane, diisopropylether, diethylether, 2-methyltetrahydrofuran, cyclopentyl methyl ether or methyl tert-butyl ether; esters such as ethyl acetate, isopropyl acetate; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, chlorobenzene, 4-chlorotoluene, trifluorotoluene, o-xylene, m-xylene or p-xylene, or mixtures thereof.

The reduction reaction time should be sufficient to complete the reaction which depends on scale and mixing procedures, as is commonly known to one skilled in the art. Typically, the reduction reaction time can vary from about few minutes to several hours. For example the reaction time can be from about 10 minutes to about 24 hours, or any other suitable time period.

The reduction of the compound of formula V to provide the compound of formula VI can take place at a temperature of about −20° C. to about 150° C., about −10° C. to about 100° C., about 0° C. to about 30° C., about room temperature, about reflux temperature of the solvent used in the reaction, or any other suitable temperature, which facilitates the desired reaction to happen without substantially negatively affecting the quality of the substrates or the reaction product.

The reaction time should be sufficient to complete the reaction which depends on scale and mixing procedures, as is commonly known to one skilled in the art. Typically, the reaction time can vary from about few minutes to several hours. For example the reaction time can be from about 10 minutes to about 24 hours, or any other suitable time period.

Optionally, the steps (c) and (d) of the process according to the first embodiment can be carried out as one-pot without isolation of the product of step (c). Optionally, the steps (c′) and (d′) of the process according to the fourth embodiment can be carried out as one-pot without isolation of the product of step (c′). Optionally, the compound of formula IV can be converted in to the compound of formula VI in presence of reducing agent with or without, an acid, or a reagent capable of releasing an acid in situ. The acid, the reagent capable of releasing an acid in situ, the reducing agent, solvent and reaction conditions of one-pot reaction are same as described respectively for steps (c) and (d) of first embodiment or steps (c′) and (d′) of the fourth embodiment.

In the present invention, the compound of formula VI can be subjected to resolution to provide corresponding enantiomerically enriched compounds of formula VII and VIIa respectively. The said resolution can be any conventional resolution method known in the art. For example, such resolution includes crystallization of enantiomer mixtures; mechanical separation of enantiomers, wherein process can be carried out under thermodynamic control or kinetic control; chemical separation of enantiomers, wherein the process can be carried out by conversion to diastereomers under thermodynamic control or kinetic control, or by intervention of diastereomeric transition states or excited states. A racemate can interact with the resolving agent by bond formation, by formation of diastereomeric complexes or by formation of diastereomeric salts.

Optionally, the compound of formula VI is subjected to optical resolution by diasteromeric salt formation followed by separation of diatereomeric salts. The optical resolution of the compound of formula VI can be carried out using an appropriate chiral resolving agent to provide corresponding diastereomeric mixture. The said diastereomeric mixture can be separated by conventional methods, which can be converted to the corresponding R- and S-isomers of the compound of formula VI or the compound of formula VIa depending on the isomer that would be required for the downstream process to get a desired isomer of suvorexant (Formula I) or enantiomer of suvorexant (Formula Ia).

For the purpose of obtaining the enantiomerically enriched compound of formula VII, that is, R-isomer, the process includes optical resolution using chiral resolving agent. The chiral resolving agent that can be used includes, (+)-2,3-Dibenzoyl-L-tartaric acid, Di-p-toluoyl-D(+)-tartaric acid, 1(R)-Camphor Sulfonic acid, L(+)-tartaric acid, L(+)-mandelic acid, L(+)-malic acid or their enantiomers or any other suitable chiral acid. For the purpose of obtaining the enantiomerically enriched compound of formula VIIa, that is, S-isomer, the process includes optical resolution using chiral resolving agent. The chiral resolving agent that can be used includes the ones described above.

The resulting diastereomeric salt mixture can be subjected to separation methods such as fractional crystallization method or chiral column chromatography to provide a mixture that is enriched with desired diastereomer. The said mixture that is enriched in the desired diastereomer can be converted in to a mixture that is enriched in one of the desired enantiomers, that is, the compound of formula VII or the compound of formula VIIa by conventional methods known in the art such as treatment with a base.

The “fractional crystallization method” includes a method in which a salt is formed between a racemate and a chiral resolving agents, which salt is separated by fractional recrystallization, and, if desired, subjecting to a neutralization process, to give a free optical isomer.

The “chiral column method” includes a method in which a racemate or a salt thereof is loaded on to a chiral column and the isomers are separated by chromatography.

Optionally, the optical purity of the compound of formula VII or the compound of formula VIIa can be enriched by any purification methods known in the art. The said method includes fractional crystallization or chiral column chromatography.

Optionally, the compound of formula V can be subjected to enantioselective reduction in presence of a suitable catalytic system to provide enantiomrically enriched compound of formula VII or enantiomerically enriched compound of formula VIIa. The said enantioselective reduction may be carried out via catalytic hydrogenation reaction or via catalytic hydrogen transfer reaction. In either case, the reduction is generally carried out in presence of a suitable chiral catalyst. Suitable chiral catalyst that can be used contains a transition metal selected from Ir, Rh, Ru, Pd or any other suitable metal and one or more ligands containing phosphorous or nitrogen. The said catalytic system that can used, include those mentioned in U.S. Pat. No. 7,816,533, U.S. Pat. No. 6,184,381 and U.S. Pat. No. 6,528,687, which are incorporated herein by reference. Optionally, in case of catalytic hydrogen transfer reduction, the reducing agent can be a boron containing agent such as α-pinene-based organoboranes including (+) or (−)-DIP-Cl (B-chlorodiisopinocampheylborane), B-chlorodiiso-2-ethylapopino-campheylborane, alpine-Borane, NB-enantride, or diisopinocampheylborane; chiral dialkoxyborane or the like.

Optionally, the compound of formula V can be subjected to enantioselective reduction in presence of an appropriate enzyme to provide the enantiomerically enriched compound of formula VII or the enantiomerically enriched compound of formula VIIa. Suitable enzyme that can used, include those mentioned in US patent publication number 2011/0287494, which is incorporated herein by reference.

The optical resolution of the compound of formula VI or enantioselective reduction of a compound of formula V can be carried out in the presence or absence of a solvent. The solvent that can be used for optical resolution of the compound of formula VI or enantioselective reduction of the compound of Formula V includes, water; C₁-C₁₀ straight or branched chain alcohol such as methanol, ethanol, isopropyl alcohol, 1-butanol, 2-butanol, 2-methyl-2-propanol, 1-pentanol, 2-pentanol, 2,2-dimethyl-1-propanol, 2,2,2-trimethyl ethanol, 1-decanol, benzyl alcohol; ethers such as tetrahydrofuran, dioxane, diisopropylether, diethylether, 2-methyltetrahydrofuran, cyclopentyl methyl ether or methyl tert-butyl ether; esters such as ethyl acetate, isopropyl acetate; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, chlorobenzene, 4-chlorotoluene, trifluorotoluene, o-xylene, m-xylene or p-xylene, or mixtures thereof.

The optical resolution of the compound of formula VI can take place at a temperature of about −20° C. to about 80° C., about 10° C. to about 50° C., about 20° C. to about 40° C., about room temperature, or any other suitable temperature.

The enantioselective reduction of the compound of formula V can take place at a temperature of about −20° C. to about 80° C., about −10° C. to about 50° C., about −5° C. to about 20° C., about 0° C. to about 5° C., about room temperature, or any other suitable temperature.

The reaction time should be sufficient to complete the reaction which depends on scale and mixing procedures, as is commonly known to one skilled in the art. Typically, the reaction time for optical resolution or the enantioselective reduction may vary from about few minutes to several hours. For example the reaction time may be from about 10 minutes to about 24 hours or any other suitable time period.

In the present invention, the compound of formula VII or the compound of formula VIIa is reacted with the compound of formula VIII to provide the compound of formula IX or the compound of formula IXa, respectively.

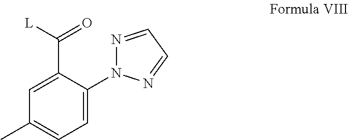

L in compound of formula VIII is a leaving group which includes, halo (Cl, Br, I); hydroxy; C₁-C₆ alkoxy such as methoxy, ethoxy, propyloxy, isopropyloxy, butyloxy, sec-butyloxy, ter-butyloxy; C₅-C₁₀ aryloxy such as phenoxy, naphthyloxy; aralkyloxy such as benzyloxy; alkanoate such as acetate, propanoate, butanoate, isobutyrate; aryloate such as benzoate, naphthoate; alkyl sulphonyloxy such a mesyloxy, ethane suphonyloxy; arylsulphonyloxy such as p-tolylsulfonate, p-nitrobenzenesulfonates; a substituted or unsubstituted or cyclic or acyclic amino that can form amide bond.

The compound of formula VIII can be prepared by any process known in the art. For example the compound of formula VIII can be prepared by a process known in the PCT publication WO 2008/147518 A1, which incorporated herein by reference.

The reaction of the compound of formula VII or the compound of formula VIIa with the compound of formula VIII can be carried out in presence of a base. Suitable base can be used includes; organic base such as DBU (1,8-diazabicyclo[5.4.0]undec-7-ene), DBN (1,5-Diazabicyclo[4.3.0]non-5-ene), DABCO (1,4-diaza-bicyclo[2.2.2]octane), ABCO (1-azabicyclo[2,2,2]octane), TBD (1,5,7-Triazabicyclo[4.4.0]dec-5-ene), DMAP (4-dimethylaminopyridine), TEA (Triethyl amine), DIPEA (N,N-diisopropylethylamine), DIEA (Diethylamine), N-methyl morpholine, lutidine, pyridine or collidine; or an inorganic base includes, hydroxides of alkali metals such as sodium hydroxide, lithium hydroxide or potassium hydroxide; carbonates of alkali metals such as sodium carbonate or potassium carbonate; bicarbonates of alkali metals such as sodium bicarbonate or potassium bicarbonate.

When L is halo, suitable halogenating agent that can be used for the conversion of the corresponding acid of the compound of formula VIII to a compound of formula VIII includes thionyl chloride, oxalyl chloride, phosphorus trichloride, phosphorus oxychloride, phosphorus pentachloride, thionyl bromide, phosphorus tribromide or phosphorus pentabromide.

Optionally, the racemic compound of formula VI can be carried forward for the reaction with the compound of formula VIII and further steps of scheme I to obtain corresponding racemic suvorexant (Formula Ib) as the final compound according to the conditions described herein after.

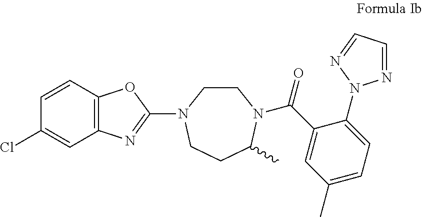 [SMILES: Cc1ccc(-n2nccn2)c(C(=O)N2CCN(c3nc4cc(Cl)ccc4o3)CCC2C)c1]

The conversion of the compound of formula IX to the compound of formula X or the conversion of the compound of formula IXa to the compound of formula Xa can be carried out in presence of palladium based catalyst such as palladium on carbon (Pd/C); by catalytic hydrogenation reaction using hydrogen gas or hydrogen transfer reagent such as formic acid, ammonium formate or phosphoric acid. Palladium on carbon (Pd/C) can be either dry or wet, wet with water up to about 50% w/w. 5% or 10% Palladium on carbon can be used.

The conversion of the compound of formula IX to the compound of formula X or the conversion of the compound of formula IXa to the compound of formula Xa can be carried out in presence of a solvent. The suitable solvent that can be used includes water; C₁-C₁₀ straight or branched chain alcohol such as methanol, ethanol, isopropyl alcohol, 1-butanol, 2-butanol, 2-methyl-2-propanol, 1-pentanol, 2-pentanol, 2,2-dimethyl-1-propanol, 2,2,2-trimethyl ethanol, 1-decanol, benzyl alcohol; ethers such as tetrahydrofuran, dioxane, diisopropylether, diethylether, 2-methyltetrahydrofuran, cyclopentyl methyl ether or methyl tert-butyl ether; esters such as ethyl acetate, isopropyl acetate; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, chlorobenzene, 4-chlorotoluene, trifluorotoluene, o-xylene, m-xylene or p-xylene, or mixtures thereof.

The reaction of the compound of formula X with the compound of formula XI to provide Suvorexant of formula I or the reaction of the compound of formula Xa with the compound of formula XI to provide Suvorexant of formula Ia can be carried out in presence of a transition metal catalyst such as copper catalyst, which includes Cu(OAc)₂, CuCl₂ or CuBr₂; RuCl₃; Pd(OAc)₂ or any other suitable catalyst.

Optionally, the reaction of the compound of formula X with the compound of formula XI to provide Suvorexant of formula I or the reaction of the compound of formula Xa with the compound of formula XI to provide Suvorexant of formula Ia can be carried out in presence of an acid, which includes acetic acid, benzoic acid, 4-nitrobenzoic acid, or 4-methoxybenzoic acid.

Optionally, the reaction of the compound of formula X with the compound of formula XI to provide Suvorexant of formula I or the reaction of the compound of formula Xa with the compound of formula XI to provide Suvorexant of formula Ia can be carried out in presence of oxygen or a reagent which can provide oxygen. The compound which can provide oxygen includes magnesium peroxide.

The molar fraction (mol %) of the copper catalyst per mole of the compound of formula XI can be from about 10 mol % to 100 mol %, about 10 mol % to about 80 mol %, about 10 mol % to about 60 mol %, about 10 mol % to about 40 mol %, or any suitable molar fraction.

The mole ratio of a compound of formula XI to a compound of formula X can be from about 1:1 to about 1:5, about 1:1 to about 1:2, about 1:1 to about 1:1.5, or about 1:1.

In a thirteenth embodiment, the present invention provides a process for preparing the compound of formula XXI:

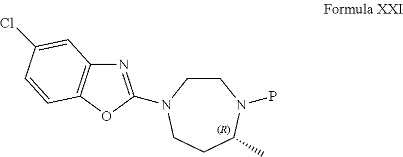

comprising:
(d) protecting the compound of formula VII to provide the compound of formula XXII:

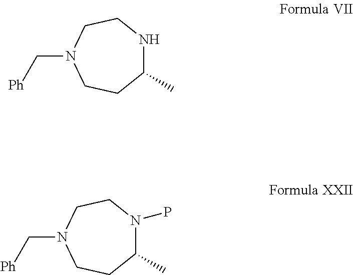

(e) converting the compound of formula XXII to obtain the compound of formula XXIII;

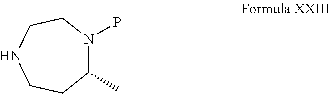

(f) reacting the compound of formula XXIII with the compound of formula XI:

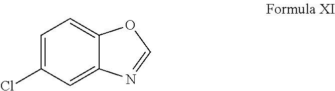

to provide a compound formula XXI.

P in compound XXII and compound XXIII represents an amino-protecting group. N-protecting group includes, aryloxycarbonyl such as benzyloxycarbonyl (Cbz), fluorenylmethoxycarbonyl (Fmoc); alkoxycarbonyl such as methyloxycarbonyl, acetoxycarbonyl, propoxycarbonyl, tert-butyloxycarbonyl (Boc); acyl such as acetyl, propanoyl, iso-butyryl, ter-butyryl, t-butylacetyl, pivaloyl; aroyl groups such as benzoyl; silyl such as trimethylsilyl, ter-butyldimethylsilyl; sulphonyl such as methanesulphonyl, p-tolylsulphonyl; sulphenyl such as 2-nitorphenylsulfenyl; urea; urethane; nitroso; nitro and the like. A thorough discussion of amino-protecting groups disclosed in Protective Groups in Organic Synthesis, Fourth edition, Wiley, New York 2006 by T. W. Greene and P. G. M. Wuts, which is incorporated herein by reference.

The compound of formula XXII, which is represented by the formula of —NHP, wherein P represents amino protecting group as described above. The compound of formula XXII can be prepared using amino protecting reagent. The term “amino-protecting reagent” as used herein refers to a compound that reacts with the amino functionality to give a protected amino group. For example, tert-butyloxycarbonyl protection can be prepared using Di-tert-butyl dicarbonate in presence of base. Similarly other amino protecting group can incorporated using amino-protecting reagents such as acylating reagents, sulfonylating reagents, sulfenylating reagents, urea and urethane-type reagents, nitroso derivatives, nitro derivatives, or silyl reagents. Various amino-protecting reagents have been described by Greene & Wuts in Protective Groups in Organic Synthesis. Person skilled in the art can choose individual reagent or reagent combinations based on desired protecting group. The reaction conditions for incorporation of protecting group can be optimized depending upon factors as the solubility of reagents, reactivity of reagents, preferred temperature ranges and suitable conditions for removing excess protecting reagent.

The amount of the amine-protecting reagent can vary depending on which amine-protecting reagent is used. Typically, the reaction can be accomplished with from about 1.0 to about 4.0 molar equivalents of the amino-protecting reagent relative to one molar equivalent of unprotected amine. Preferably, about 1.0 to about 1.5 molar equivalents of the amino-protecting reagent can be used. The reaction can be accomplished in the presence of an organic or inorganic base.

In step (b) of the thirteenth embodiment, the compound of formula XXII is converted to the compound of formula XXIII. The said conversion can be carried out in presence of palladium based catalyst such as palladium on carbon (Pd/C); by catalytic hydrogenation reaction using hydrogen gas or hydrogen transfer reagent such as formic acid, ammonium formate or phosphoric acid. Palladium on carbon (Pd/C) can be either dry or wet, wet with water up to about 50% w/w. 5% or 10% Palladium on carbon can be used.

The conversion in step (b) of the thirteenth embodiment can be carried out in presence of a solvent. The suitable solvent that can be used in step (b) includes C₁-C₁₀ straight or branched chain alcohol such as methanol, ethanol, isopropyl alcohol, 1-butanol, 2-butanol, 2-methyl-2-propanol, 1-pentanol, 2-pentanol, 2,2-dimethyl-1-propanol, 2,2,2-trimethyl ethanol, 1-decanol, benzyl alcohol; ethers such as tetrahydrofuran, dioxane, diisopropylether, diethylether, 2-methyltetrahydrofuran, cyclopentyl methyl ether or methyl tert-butyl ether; esters such as ethyl acetate, isopropyl acetate; halogenated solvents such as dichloromethane, chloroform, tetrachloromethane, dichloroethane, chlorobenzene or dichlorobenzene; aliphatic hydrocarbon solvents such as methylcyclohexane, cyclohexane, heptane or hexane; aromatic hydrocarbon solvents such as toluene, benzene, chlorobenzene, 4-chlorotoluene, trifluorotoluene, o-xylene, m-xylene or p-xylene, or mixtures thereof.

In step (c) of the thirteenth embodiment, the reaction of the compound of formula XXIII with a compound of formula XI to obtain the compound of formula XXI can be carried in presence of a transition metal catalyst such as copper catalyst, which includes Cu(OAc)₂, CuCl₂ or CuBr₂; RuCl₃; Pd(OAc)₂ or any other suitable catalyst.

Optionally the process according to step (c) of the thirteenth embodiment can be carried out in presence of an acid, which includes acetic acid, benzoic acid, 4-nitrobenzoic acid or 4-methoxybenzoic acid.

Optionally, the process according to step (c) of the thirteenth can be carried out in presence of oxygen or a reagent which can provide oxygen, the compound which can provide oxygen includes magnesium peroxide.

The molar fraction (mol %) of the copper catalyst per mole of the compound of formula XI can be from about 10 mol % to 100 mol %, about 10 mol % to about 80 mol %, about 10 mol % to about 60 mol %, about 10 mol % to about 40 mol %, or any suitable molar fraction.

The mole ratio of a compound of formula XI to a compound of formula XXIII can be from about 1:1 to about 1:5, about 1:1 to about 1:2, about 1:1 to about 1:1.5, or about 1:1 molar equivalent.

Optionally, a compound of formula XXI obtained by a process of the present invention can be used to prepare suvorexant of formula I according to a process according to the present invention or any method known in the art, for example, by a method reported in the PCT publication WO 2012/148553A1.

In a fourteenth embodiment, the present invention provides a process for the preparation of a compound of formula XXV, which comprises:

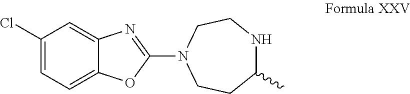 [SMILES: CC1CCN(c2nc3cc(Cl)ccc3o2)CCN1]

converting the compound of formula XXIV to the compound of formula XXV

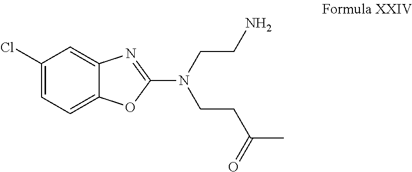 [SMILES: CC(=O)CCN(CCN)c1nc2cc(Cl)ccc2o1]

in presence of a reagent and a reducing agent.

The reagent which can be used in the said conversion in fourteenth embodiment includes an acid or a reagent capable of releasing an acid in situ. The acid or the reagent capable of releasing an acid in situ can selected from reagent described in step (c) of first embodiment of the present invention, which is incorporated herein by reference.

The reducing agent which can be used can be selected from the reducing agent described for the reduction of the compound of formula V to obtain the compound of formula VI of the present invention, as described herein before.

The said conversion of the compound of formula XXV in the fourteenth embodiment passes through the following intermediate up on treatment of a compound of formula XXIV with a reagent as defined herein above.

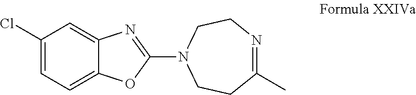 [SMILES: CC1=NCCN(c2nc3cc(Cl)ccc3o2)CC1]

The said compound of Formula XXIVa subsequently gets reduced to a compound of formula XXV with the reducing agent defined herein above. Optionally, the compound of the formula XXIVa is isolated and purified.

Optionally, the process according to the fourteenth embodiment can be carried out as one-pot without isolation of the compound of the formula XXIVa. Optionally, the compound of formula XXIVa can be converted in to the compound of formula XXV in presence of reducing agent with or without, an acid, or a reagent capable of releasing an acid in situ.

Optionally the said reduction of a compound of formula XXIV may be affected enantioselectively to provide a compound of formula XXVa (R-isomer) or XXVb (S-isomer):

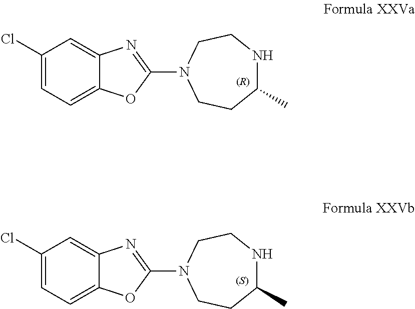

The said enantioselective reduction of the compound of formula XXIVa may be affected under the similar conditions described for the enantioselective reduction of the compound of formula V herein above.

Optionally, a compound of formula XXV or the compound of formula XXVa obtained by a process of the present invention can be used to prepare suvorexant (formula I) and the compound of formula XXVb obtained by a process of the present invention can be used to prepare enantiomer of suvorexant (formula Ia) respectively by following a suitable process according to the present invention or any suitable method known in the art, for example, by a method similar to that reported in the PCT publication WO 2012/148553 A1.

Certain specific aspects and embodiments of the present invention will be explained in more detail with reference to the following examples, which are provided for purposes of illustration only and should not be construed as limiting the scope of the present invention in any manner.

### EXAMPLE 1

### Preparation of 5-Chloro-1,3-benzoxazole (Formula XI)

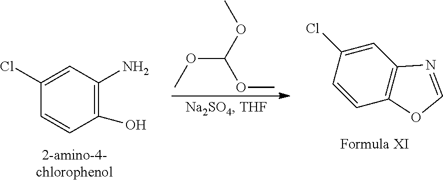

2-amino-4-chloro phenol (50 g, 0.349 moles), Trimethyl orthoformate (111 g, 1.048 moles), sodium sulphate (9.93 g, 0.069 moles) and THF (500 mL) were charged in round bottom flask at room temperature (RT) under inert atmosphere. Reaction mass was heated to 55° C. to 60° C. The progress of the reaction was monitored by TLC. After completion of the reaction, reaction mixture was cooled to room temperature and quenched with water (50 ml). Reaction mass was cooled to RT, diluted with water (250 ml) and extracted with ethyl acetate (2×250 ml). The combined organic extracts were dried over sodium sulphate, filtered and concentrated under reduced pressure. The obtained crude material was purified by column chromatography using silica gel (60-120 mesh) eluted with 20% EtOAc-hexane to give 5-Chloro-1,3-benzoxazole (Formula XI, 50 g, yield 95%, HPLC purity of 99.46%) as a yellow color solid.

### EXAMPLE 2

### Preparation of tert-butyl(2-(benzylamino)ethyl)carbamate (Formula II)

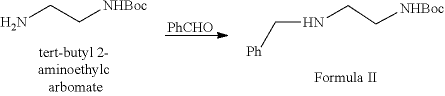 [SMILES: "PhCHO" ; CC(C)(C)OC(=O)NCCN ; CC(C)(C)OC(=O)NCCNCc1ccccc1]

Tert-butyl 2-aminoethylcarbamate (100 g, 0.625 moles) and methanol (500 ml) were charged into round bottom flask at room temperature and stirred to get pale yellow colored clear solution. Reaction mixture was cooled to 0° C.-10° C. and sodium sulphate (25 g, 0.187 moles) was added to the reaction mixture. Benzaldehyde (53 g, 0.5 moles) was added to the reaction mixture portion wise over a period of 20-30 min at 0° C.-10° C. and stirred at room temperature. The progress of the reaction was monitored by TLC. After completion of the reaction, the reaction mixture was cooled to 0° C.-10° C. then sodium borohydride was added portion wise over a period of 20-30 min at the same temperature and stirred at RT for 4 h. After completion of the reaction, the reaction mixture was cooled to 0° C.-10° C., quenched with 30% Citric acid aqueous solution (300 ml) and stirred for 20-30 min at room temperature. The reaction mixture was concentrated under reduced pressure at 40-45° C. to obtain pale yellow turbid solution, diluted with ethyl acetate (250 ml) and stirred for 30 min at room temperature. Both aqueous and organic layers were separated and aqueous layer was again extracted with ethyl acetate (250 ml). The aqueous layer was basified with saturated sodium bicarbonate solution and again extracted with ethyl acetate (2×300 ml). The organic extracts were combined, washed with brine solution (200 ml), dried over sodium sulfate, filtered and concentrated under reduced pressure at 45-50° C. to obtain tert-butyl (2-(benzylamino)ethyl)carbamate (Formula II, 110 g, yield 70.69%, HPLC purity of 93.38%) as a pale yellow color liquid.

### EXAMPLE 3

### Preparation of tert-butyl(2-(benzyl(3-oxobutyl)amino)ethyl)carbamate (Formula III)

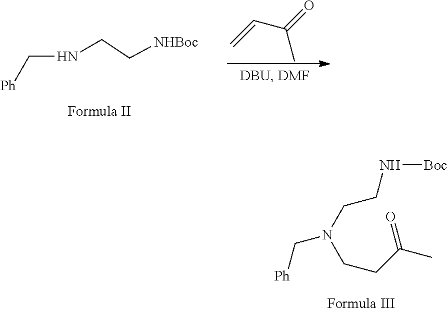

Tert-butyl(2-(benzylamino)ethyl)carbamate (Formula II, 50 g, 0.200 moles), 1,8-Diazabicycloundec-7-ene (61.12 ml, 0.401 moles) and DMF (300 ml) were charged in round bottom flask at RT and stirred to get heterogeneous solution. The reaction mixture was cooled to 0-5° C. and methyl vinyl ketone (75.3 ml, 0.903 moles) was added to the reaction mixture slowly over a period of 30 min. The resulting reaction mixture was stirred at room temperature for 16 h. After completion of the reaction, the reaction mixture was cooled to 0-5° C. and diluted with water (500 ml). The aqueous solution was extracted with ethyl acetate (250 ml×2). The organic layer was washed with 1N HCL (200 ml). The pH of aqueous layer was adjusted to 8 with saturated NaHCO₃ solution (200 ml) and extracted with ethyl acetate (250 ml×2). The combined organic layers were dried over sodium sulfate, filtered and concentrated under reduced pressure at 45-50° C. to give tert-butyl(2-(benzyl(3-oxobutyl)amino)ethyl)carbamate (Formula III, 30 g, yield 46.87%) as a dark brown color liquid.

### EXAMPLE 4

### Preparation of 4-((2-aminoethyl)(benzyl)amino)butan-2-one (Formula IV)

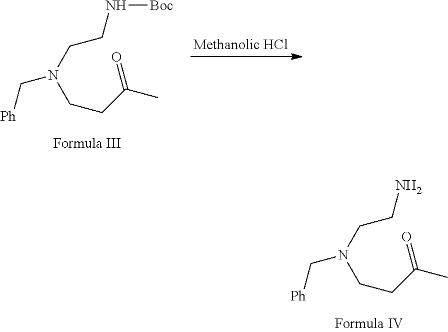 [SMILES: CC(=O)CCN(CCNC(=O)OC(C)(C)C)Cc1ccccc1 ; CC(=O)CCN(CCN)Cc1ccccc1]

Tert-butyl(2-(benzyl(3-oxobutyl)amino)ethyl)carbamate (Formula III, 30 g, 0.093 moles) and methanolic HCl (150 ml) were charged in round bottom flask at 10-15° C. and stirred at room temperature for 6 h. After completion of the reaction, the reaction mixture was concentrated under reduced pressure. The obtained crude material was diluted with ice-cold water (150 ml) and extracted with DCM (300 ml). The pH of aqueous layer was adjusted 8 with saturated NaHCO₃ solution (300 ml) and extracted with DCM (300 ml×2). The combined organic layers were dried over Na₂SO₄ and concentrated under reduced pressure to give 4-((2-aminoethyl)(benzyl)amino)butan-2-one (Formula IV, 17 g, crude) as dark brown color syrup. The obtained crude compound was taken up for the next step without any further purification.

### EXAMPLE 5

### Preparation of (rac)-1-benzyl-5-methyl-1,4-diazepane (Formula VI)

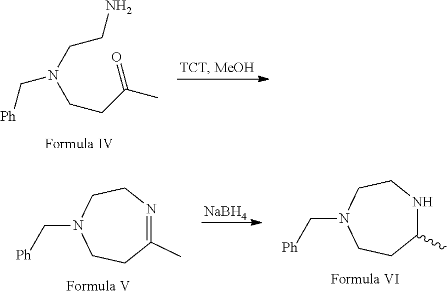 [SMILES: CC(=O)CCN(CCN)Cc1ccccc1 ; CC1=NCCN(Cc2ccccc2)CC1 ; "NaBH4" ; CC1CCN(Cc2ccccc2)CCN1]

4-((2-aminoethyl)(benzyl)amino)butan-2-one (Formula IV, 17 g, 0.077 moles), cyanuric chloride (0.6 g, 0.043 moles) and methanol (170 ml) were charged were charged in round bottom flask under nitrogen atmosphere at 0-5° C. The temperature of reaction mixture was raised to room temperature and stirred for 16 h. Sodium borohydride (2.93 g, 0.077 moles) was added to the reaction mixture in portions over a period of 30 min at 0-5° C. The temperature of reaction mixture was raised to room temperature and stirred for 1 h. After completion of the reaction, the reaction mixture was quenched with ice-cold water (100 ml) at 5-10° C.; temperature was raised to RT and concentrated under reduced pressure at 45-50° C. The reaction mixture was extracted with EtOAc (2×170 ml). The combined organic layers were washed with brine solution, dried over Na₂SO₄ and concentrated under reduced pressure to give 1-benzyl-5-methyl-1,4-diazepane (Formula VI, 15 g) as pale brown syrup.

### EXAMPLE 6

### Preparation of (R)-1-benzyl-5-methyl-1,4-diazepane (Formula VII)

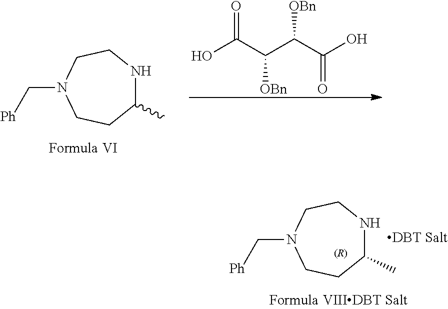

(rac)-1-benzyl-5-methyl-1,4-diazepane (Formula VI, 15 g, 0.073 moles) and THF (40 ml) were charged in round bottom flask at room temperature. (+) 2,3-Dibenzoyl-D-Tartaric acid (44.78 g, 0.124 moles) in THF (35 ml) was added to the reaction mixture at 5-10° C. The temperature of reaction mixture was raised to room temperature and stirred for 16 h. Methyl tert-butyl ether (100 ml) was added to the reaction mixture at 5-10° C. and stirred for 1 h. The obtained solid was filtered, washed with Methyl tert-butyl ether (50 mL) and dried under vacuum to furnish (R)-1-benzyl-5-methyl-1,4-diazepane (+)-2,3-Dibenzoyl-D-Tartarate salt (Formula VII.DBT salt, 30 g) as an off white solid.

### EXAMPLE 7

### Preparation of (R)-(4-benzyl-7-methyl-1,4-diazepan-1-yl)(5-methyl-2-(2H-1,2,3-triazol-2-yl)phenyl)methanone (Formula IX)

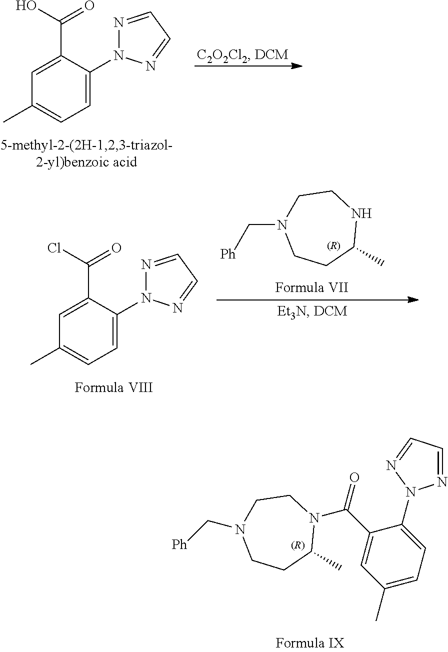

5-methyl-2-(2H-1,2,3-triazol-2-yl)benzoic acid (11.94 gm, 0.058 moles) and DCM (15 ml) were charged in round bottom flask at room temperature. The reaction mixture was cooled to 0-5° C. and oxalyl chloride (5.07 mL, 0.058 moles) was added under inert atmosphere followed by the addition of DMF (1.18 mL, 0.0152 moles). The temperature of reaction mixture was raised to RT and stirred for 2 h. (R)-1-benzyl-5-methyl-1,4-diazepane (12 g, 0.058 moles, formula VII. DBT salt was neutralized with NaOH to obtain free base), triethyl amine (16.57 mL, 0.1176 moles) and DCM (500 mL) were taken in another RBF at 0-5° C. and stirred at room temperature for 2 h. The resulting mixture was slowly added to mixture containing triazole at 0-5° C. and stirred. The progress of the reaction was monitored by TLC. After completion of the reaction, the reaction mixture was diluted with water and extracted with DCM (2×7.5 mL). The combined organic layer was dried over Na₂SO₄ and concentrated under reduced pressure to give crude material. The obtained crude material was purified by column chromatography using silica gel (60-120 mesh), 2% MeOH-DCM as an eluent to give (R)-(4-benzyl-7-methyl-1,4-diazepan-1-yl)(5-methyl-2-(2H-1,2,3-triazol-2-yl)phenyl)methanone (Formula IX, 18 g) as a brown color liquid. The obtained crude compound was used for the next step without any further purification.

### EXAMPLE 8

### Preparation of (R)-(7-methyl-1,4-diazepan-1-yl)(5-methyl-2-(2H-1,2,3-triazol-2-yl)phenyl)methanone (Formula X)

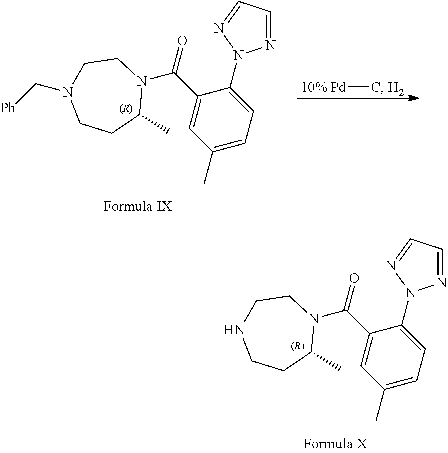

(R)-(4-benzyl-7-methyl-1,4-diazepan-1-yl)(5-methyl-2-(2H-1,2,3-triazol-2-yl)phenyl)methanone (Formula IX, 18 g, 0.462 moles), 10% Pd/C (5 g) and methanol (144 mL), were charged in a steel hydrogenation vessel at room temperature. The resulting reaction mixture was hydrogenated using parr hydrogenator (H₂, 70 Psi) at room temperature for 16 h. The progress of the reaction was monitored by TLC. After completion of the reaction, the reaction mixture was filtered through celite and washed with methanol (2.5 ml). The filtrate was concentrated under reduced pressure to give (R)-(7-methyl-1,4-diazepan-1-yl)(5-methyl-2-(2H-1,2,3-triazol-2-yl)phenyl)methanone (Formula X, 11 g, crude) as pale brown syrup. The obtained crude material was used for next reaction without any further purification.

### EXAMPLE 9

### Preparation of Suvorexant (Formula I)

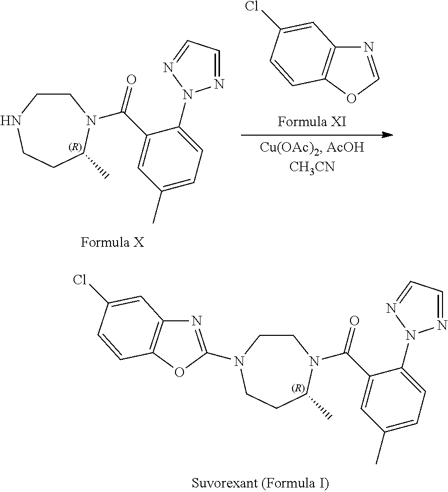

In a sealed tube, (R)-(7-methyl-1,4-diazepan-1-yl)(5-methyl-2-(2H-1,2,3-triazol-2-yl)phenyl)methanone (Formula X, 5.5 g, 0.0183 moles), 5-Chloro-1,3-benzoxazole (Formula XI, 2.8 g, 0.0183 moles), Cu(OAc)₂ (732 mg, 0.0036 moles), acetic acid (2.2 mL, 0.036 moles) and acetonitrile (55 mL) were charged at room temperature. Reaction mixture was purged with oxygen gas for 5 min. The reaction mixture was heated to 70° C. and stirred for 4 h. The progress of the reaction was monitored by TLC. After completion of the reaction, the reaction mixture was cooled to RT, concentrated under reduced pressure and diluted with saturated sodium bicarbonate solution (55 ml). The aqueous solution was extracted with DCM (2×50 ml). The combined organic extracts were dried over sodium sulphate and concentrated under reduced pressure. The obtained crude material was purified by column chromatography using silica gel (60-120 mesh), 2% MeOH-DCM as eluent to give suvorexant (Formula I, 3.3 g, chiral purity by HPLC 99.96%) as a white solid.

### EXAMPLE 10

### Preparation of tert-butyl(R)-4-benzyl-7-methyl-1,4-diazepane-1-carboxylate (Formula XXII)

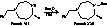 [SMILES: "Boc2O" ; C[C@@H]1CCN(Cc2ccccc2)CCN1 ; C[C@@H]1CCN(Cc2ccccc2)CCN1C(=O)OC(C)(C)C]

In a 100 mL single neck RBF charged (R)-1-benzyl-4,5-dimethyl-1,4-diazepane (Formula VII, 1 g, 0.004 moles), Boc₂O (1.3 mL, 0.005 moles), TEA (1.3 mL, 0.009 moles), and methanol were charged in round bottom flask at room temperature under inert atmosphere and stirred for 2 h. After completion of the reaction, reaction mixture was concentrated under reduced pressure and diluted with water (50 ml). The aqueous layer was extracted with DCM (2×10 ml). The organic layer was washed with 5% Citric acid solution (50 ml), dried over sodium sulphate, filtered and concentrated under reduced pressure. The obtained crude material was purified by column chromatography using silica gel (60-120 mesh) and 2% MeOH-DCM as eluent to give tert-butyl(R)-4-benzyl-7-methyl-1,4-diazepane-1-carboxylate (Formula XXII, 1.1 g, 78.5%) as yellow syrup.

### EXAMPLE 11

### Preparation of tert-butyl(R)-7-methyl-1,4-diazepane-1-carboxylate (Formula XXIII)

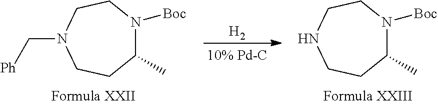 [SMILES: C[C@@H]1CCN(Cc2ccccc2)CCN1C(=O)OC(C)(C)C ; "H2" ; C[C@@H]1CCNCCN1C(=O)OC(C)(C)C]

Tert-butyl(R)-4-benzyl-7-methyl-1,4-diazepane-1-carboxylate (Formula XXII, 1 g, 0.003 moles), 10% Pd/C and methanol (10 mL) were charged in a steel hydrogenation vessel at room temperature. Reaction mixture was hydrogenated using parr hydrogenator (H₂, 70 Psi) at room temperature for 16 h. After completion of the reaction, the reaction mixture was filtered through celite bed and washed with methanol (10 ml). The filtrate was concentrated under reduced pressure to give tert-butyl(R)-7-methyl-1,4-diazepane-1-carboxylate (Formula XXIII, 600 mg, 85.7%) as yellow syrup.

### EXAMPLE 12

### Preparation of tert-butyl(R)-4-(5-chlorobenzo[d]oxazol-2-yl)-7-methyl-1,4-diazepane-1-carboxylate (Formula XXI)

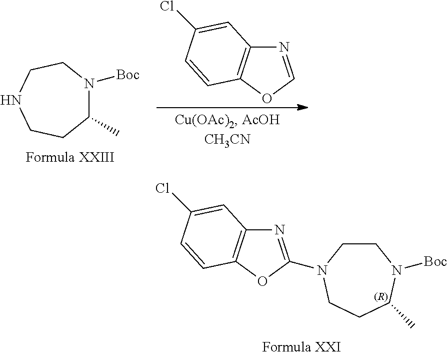

In a sealed tube, tert-butyl(R)-7-methyl-1,4-diazepane-1-carboxylate (Formula XXIII, 100 mg, 0.0004 moles), 5-Chloro-1,3-benzoxazole (Formula XI, 71 mg, 0.0004 moles), Cu(OAc)₂ (18 mg, 0.00009 moles), acetic acid (56 mg, 0.002 moles) and acetonitrile (1 ml) were charged at room temperature and purged with oxygen gas for 5 min. The reaction mixture was heated to 70° C. and stirred for 4 h. After completion of the reaction, the reaction mixture was cooled to RT, concentrated under reduced pressure and diluted with saturated sodium bicarbonate solution. The aqueous solution was extracted with DCM (2×10 ml). The combined organic extracts were dried over sodium sulphate and concentrated under reduced pressure. The obtained crude material was purified by column chromatography using silica gel (60-120 mesh), 20% EtOAc-Hexane as eluent to give tert-butyl(R)-4-(5-chlorobenzo[d]oxazol-2-yl)-7-methyl-1,4-diazepane-1-carboxylate (Formula XXI, 83.3 mg, 50%) as a yellow syrup.

### EXAMPLE 13

### Preparation of 5-Chloro-1,3-benzoxazole-2-thiol

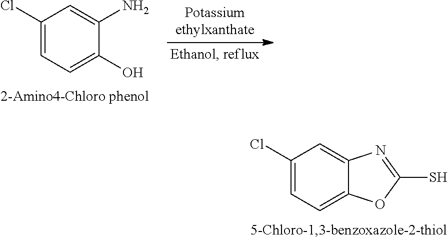 [SMILES: Nc1cc(Cl)ccc1O ; Sc1nc2cc(Cl)ccc2o1]

Potassium ethylxanthate (893.2 g, 5.572 moles) was added to a stirred mixture of 2-Amino-4-Chloro phenol (400 g, 2.786 moles) and Ethanol (3 liter) at room temperature and stirred for 10 minutes. The obtained mixture was stirred at 80° C. until reaction was complete. The progress of the reaction was monitored by TLC. After completion of the reaction, reaction mixture was poured into cold water (7 liter) and neutralized with acetic acid (752 ml). The solid was filtered and washed with cold water (5 liter), Hexane (2 liter) and dried under vacuum to furnish 5-Chloro-1,3-benzoxazole-2-thiol (510 g, 98.4%) as an off white solid.

### EXAMPLE 14

### Preparation of 2,5-dichloro-1,3-benzoxazole

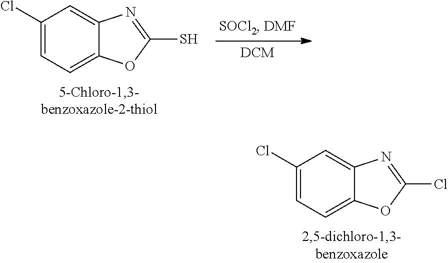 [SMILES: Sc1nc2cc(Cl)ccc2o1 ; Clc1ccc2oc(Cl)nc2c1]

Thionyl Chloride (540 mL) and Dimethyl formamide (270 mL) were added to a stirred mixture of 5-Chloro-1,3-benzoxazole-2-thiol (540 g, 2.903 moles) and dichloromethane (5 liter) at 5-10° C. and stirred till clear solution was observed. The reaction mixture was then stirred at 10° C. to room temperature for 4 hour. After completion of the reaction, the mixture was poured into cold water (4 liter), neutralized with solid sodium bicarbonate (1440 g) portion wise over a period of 1 hour and extracted with dichloromethane (2×2.5 liter). The combined organic extracts were washed with brine solution, dried over anhydrous sodium sulphate (300 g) and concentrated under reduced pressure to give a crude compound. The crude material was triturated with Hexane (2×2 liter) at −20° C., filtered and and dried under reduced pressure to obtain 2,5-dichloro-1,3-benzoxazole (475 g, 88.9%) as a yellow liquid.

### EXAMPLE 15

### Preparation of t-butyl{2-[(5-Chlorobenzoxazol-2-yl)-amino]ethyl}-carbamate

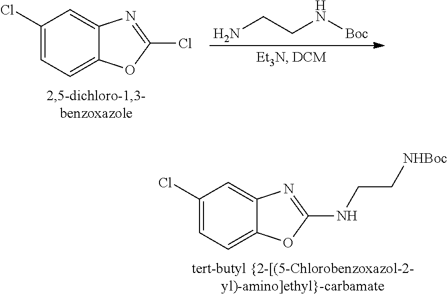 [SMILES: Clc1ccc2oc(Cl)nc2c1 ; CC(C)(C)OC(=O)NCCN ; CC(C)(C)OC(=O)NCCNc1nc2cc(Cl)ccc2o1]

A solution of triethylamine (536 ml, 3.852 mole) and N-Boc-ethylenediamine (487 ml, 3.081 mole) in dichloromethane (500 mL) was added to stirred mixture of 2,5-dichloro-1,3-benzoxazole (470 g, 2.568 mole) and dichloromethane at 5-10° C. over a period of 30 minute. The reaction mixture was then warmed to room temperature and stirred for 16 hour. The reaction mixture was diluted with water (10 liter) and extracted with dichloromethane (2×1.5 liter). The combined organic extracts were washed with brine solution and dried over anhydrous sodium sulphate. Organic layer was filtered, washed with EtOAc (2 liter) and concentrated under reduced pressure to give a crude compound. The crude material was triturated with Hexane (1.5 liter) and MTBE (500 ml), filtered and dried to furnish tert-butyl{2-[(5-Chlorobenzoxazol-2-yl)-amino]ethyl}-carbamate (666 g, 85.3%) as an off white solid.

### EXAMPLE 16

### Preparation of t-butyl{2-[(5-Chlorobenzoxazol-2-yl)-(3-oxo-butyl)-amino]ethyl}carbamate

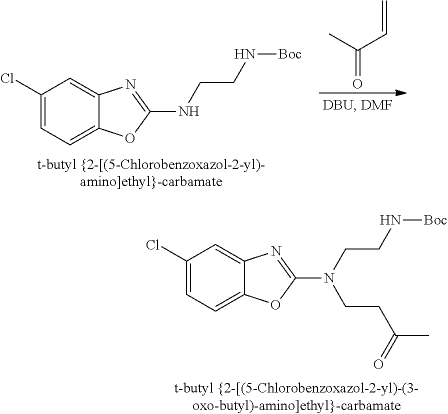

Methyl vinyl ketone (528 ml, 6.346 mole) and 1,8-Diazabicyclo[5.4.0]undec-7ene (632 ml, 4.230 mole) were added to a solution of t-butyl{2-[(5-Chlorobenzoxazol-2-yl)-amino]ethyl}-carbamate (660 g, 2.115 moles) in dimethyl formamide (6 liter) at 0-5° C. then stirred at room temperature for 16 hour. The reaction mixture was poured into cold water (10 liter) and extracted with ethyl acetate (7 liter). The combined organic extracts were washed with water (2×2 liter), dried over Na₂SO₄ and concentrated under reduced pressure to give a crude compound. The crude compound was triturated with Hexane (1.5 liter) and MTBE (500 ml), filtered and dried under vacuum to furnish t-butyl{2-[(5-Chlorobenzoxazol-2-yl)-(3-oxo-butyl)-amino]ethyl}-carbamate (386 g, 47.7%) as an off white solid.

### EXAMPLE 17

### Preparation of 4-[(2-amino-ethyl)-(5-chlorobenzoxazol-2-yl)amino]butan-2-one (Formula XXIV)

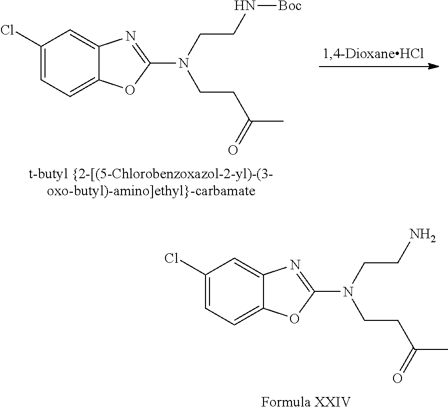 [SMILES: CC(=O)CCN(CCNC(=O)OC(C)(C)C)c1nc2cc(Cl)ccc2o1 ; CC(=O)CCN(CCN)c1nc2cc(Cl)ccc2o1]

t-butyl{2-[(5-Chlorobenzoxazol-2-yl)-(3-oxo-butyl)-amino]ethyl}-carbamate (250 g, 0.656 moles) and 1,4-Dioxane.HCl (2.5 L) were charged round bottom flask and stirred at room temperature. The progress of the reaction was monitored by TLC. After the completion of reaction, the obtained salt was filtered and washed with 1,4-dioxane, dried under vacuum. The obtained solid was neutralized with aqueous ammonium (200 ml) solution and extracted with dichloromethane (2×1 liter). The combined organic layers were washed with brine (2×500 ml), dried over Na₂SO₄ and concentrated under reduced pressure to give 4-[(2-amino-ethyl)-(5-chlorobenzoxazol-2-yl)amino]butan-2-one (171 g, crude) as a brown color thick syrup. The obtained crude material was used for the next step without any further purification.

### EXAMPLE 18

### Preparation of 5-Chloro-2-(5-methyl-[1,4]diazepan-1-yl)-benzoxazole (Formula XXV)

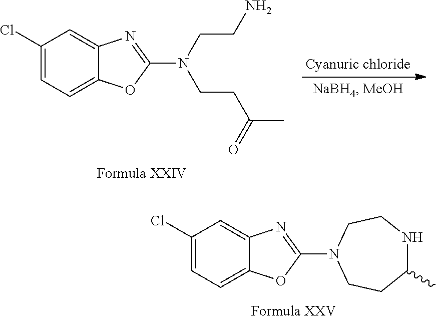 [SMILES: CC(=O)CCN(CCN)c1nc2cc(Cl)ccc2o1 ; CC1CCN(c2nc3cc(Cl)ccc3o2)CCN1]

4-[(2-Amino-ethyl)-(5-chlorobenzoxazol-2-yl)amino]butan-2-one (171 g, 0.604 mole), cyanuric chloride (4.45 g, 0.024 mole) and methanol (1.7 liter) were charged in round bottom flask under nitrogen atmosphere. The reaction mixture was stirred at room temperature for 12 hour. The mixture was then cooled to 0° C. followed by addition of sodium borohydride (22.95 g, 0.604 mole), warmed to room temperature and stirred for 2 hour. The progress of the reaction was monitored by TLC. After completion of the reaction, mixture was concentrated under reduced pressure and quenched with water (1 liter). The aqueous solution was extracted with ethyl acetate (2×1 liter) and washed with brine solution (500 ml). The organic layer was dried over anhydrous sodium sulphate and concentrated under reduced pressure to give 5-Chloro-2-(5-methyl-[1,4]diazepan-1-yl)-benzoxazole (167 g).

1. A process for preparing suvorexant (Formula I) comprising one or more from steps (a) to (h) according to synthetic scheme I: 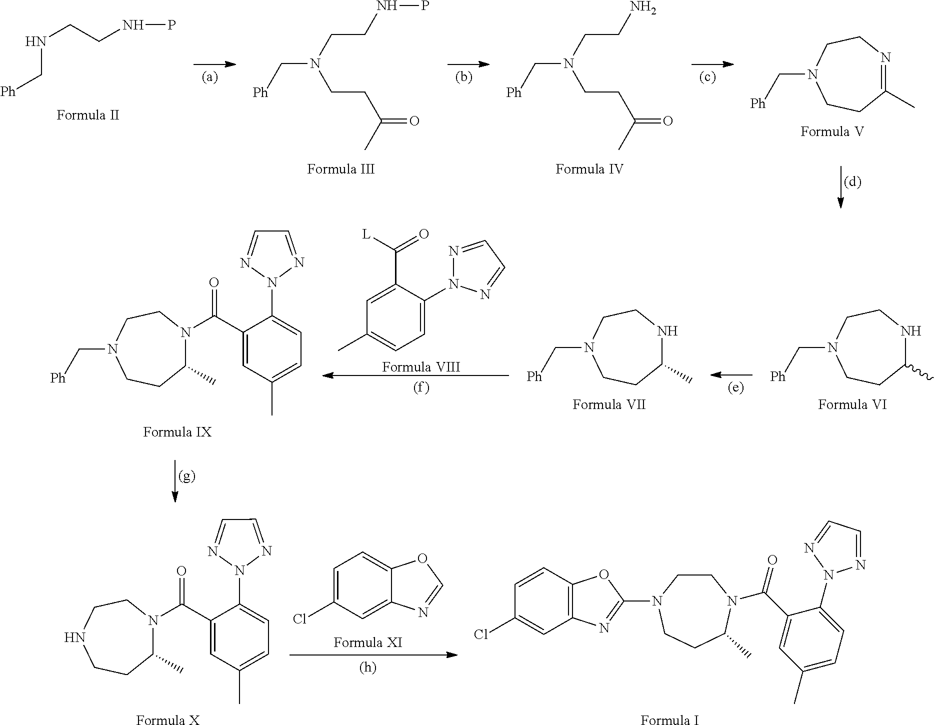
wherein P is an amino protecting group which includes, aryloxycarbonyl such as benzyloxycarbonyl (Cbz), fluorenylmethoxycarbonyl (Fmoc); alkoxycarbonyl such as methyloxycarbonyl, acetoxycarbonyl, propoxycarbonyl, tert-butyloxycarbonyl (Boc); acyl such as acetyl, propanoyl, iso-butyryl, tert-butyryl, t-butylacetyl, pivaloyl; aroyl groups such as benzoyl; silyl such as trimethylsilyl, ter-butyldimethylsilyl; sulphonyl such as methanesulphonyl, p-tolylsulphonyl; sulphenyl such as 2-nitorphenylsulfenyl; urea; urethane; nitroso; and nitro;
wherein L is a leaving group which includes, halo (Cl, Br, I); hydroxy; C₁-C₆ alkoxy such as methoxy, ethoxy, propyloxy, isopropyloxy, butyloxy, sec-butyloxy, ter-butyloxy; C₅-C₁₀ aryloxy such as phenoxy, naphthyloxy; aralkyloxy such as benzyloxy; alkanoate such as acetate, propanoate, butanoate, isobutyrate; aryloate such as benzoate, naphthoate; alkyl sulphonyloxy such a mesyloxy, ethane suphonyloxy; arylsulphonyloxy such as p-tolylsulfonate, p-nitrobenzenesulfonates; a substituted or unsubstituted or cyclic or acyclic amino that can form amide bond.

2. A process for preparing suvorexant (Formula I) comprising conversion of the compound of Formula II to the compound of formula III: 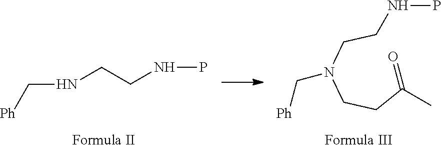
wherein P is an amino protecting group which includes, aryloxycarbonyl such as benzyloxycarbonyl (Cbz), fluorenylmethoxycarbonyl (Fmoc); alkoxycarbonyl such as methyloxycarbonyl, acetoxycarbonyl, propoxycarbonyl, tert-butyloxycarbonyl (Boc); acyl such as acetyl, propanoyl, iso-butyryl, tert-butyryl, t-butylacetyl, pivaloyl; aroyl groups such as benzoyl; silyl such as trimethylsilyl, ter-butyldimethylsilyl; sulphonyl such as methanesulphonyl, p-tolylsulphonyl; sulphenyl such as 2-nitorphenylsulfenyl; urea; urethane; nitroso; and nitro.

3. A process for preparing suvorexant (Formula I) comprising conversion of the compound of Formula III to the compound of formula IV: 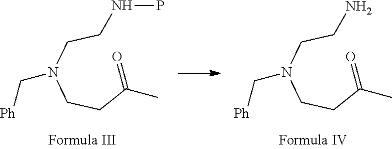
wherein P is an amino protecting group which includes, aryloxycarbonyl such as benzyloxycarbonyl (Cbz), fluorenylmethoxycarbonyl (Fmoc); alkoxycarbonyl such as methyloxycarbonyl, acetoxycarbonyl, propoxycarbonyl, tert-butyloxycarbonyl (Boc); acyl such as acetyl, propanoyl, iso-butyryl, tert-butyryl, t-butylacetyl, pivaloyl; aroyl groups such as benzoyl; silyl such as trimethylsilyl, ter-butyldimethylsilyl; sulphonyl such as methanesulphonyl, p-tolylsulphonyl; sulphenyl such as 2-nitorphenylsulfenyl; urea; urethane; nitroso; and nitro.

4. A process for preparing suvorexant (Formula I) comprising conversion of the compound of Formula IV to the compound of formula VI:

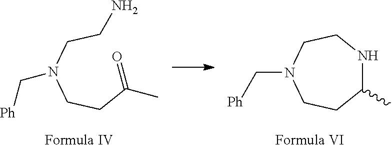 [SMILES: CC(=O)CCN(CCN)Cc1ccccc1 ; CC1CCN(Cc2ccccc2)CCN1]

5. A process for preparing suvorexant (Formula I) comprising conversion of the racemic compound of Formula VI to the compound of formula VII that is enantiomerically enriched.

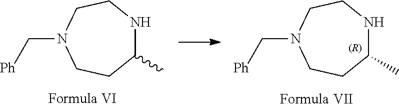

6. A process for preparing enantiomer of suvorexant (Formula Ia) comprising conversion of the racemic compound of Formula VI to the compound of formula VIIa that is enantiomerically enriched:

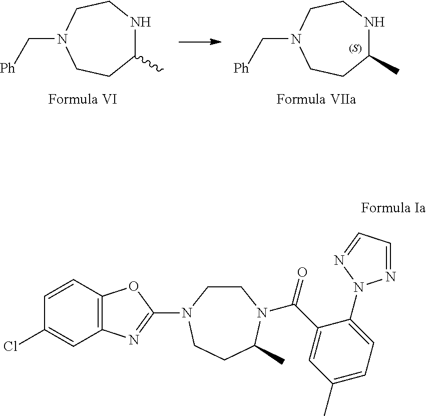

7. A process for preparing suvorexant (Formula I) comprising reaction of the enantiomerically enriched compound of Formula VII with the compound of formula VIII to provide the compound of formula IX: 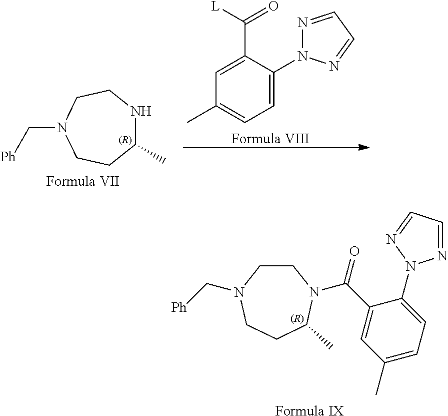
wherein L is a leaving group which includes, halo (Cl, Br, I); hydroxy; C₁-C₆ alkoxy such as methoxy, ethoxy, propyloxy, isopropyloxy, butyloxy, sec-butyloxy, ter-butyloxy; C₅-C₁₀ aryloxy such as phenoxy, naphthyloxy; aralkyloxy such as benzyloxy; alkanoate such as acetate, propanoate, butanoate, isobutyrate; aryloate such as benzoate, naphthoate; alkyl sulphonyloxy such a mesyloxy, ethane suphonyloxy; arylsulphonyloxy such as p-tolylsulfonate, p-nitrobenzenesulfonates; a substituted or unsubstituted or cyclic or acyclic amino that can form amide bond.

8. A process for preparing enantiomer of suvorexant (Formula Ia) comprising reaction of the enantiomerically enriched compound of Formula VIIa with the compound of formula VIII to provide the compound of formula IXa: 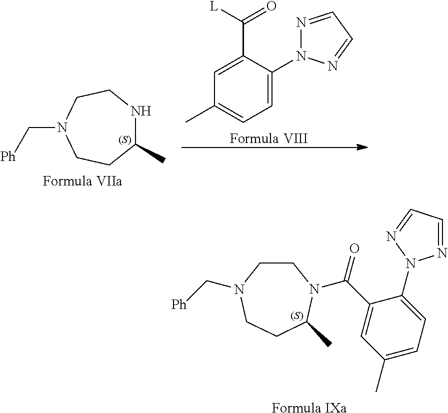
wherein L is a leaving group which includes, halo (Cl, Br, I); hydroxy; C₁-C₆ alkoxy such as methoxy, ethoxy, propyloxy, isopropyloxy, butyloxy, sec-butyloxy, ter-butyloxy; C₅-C₁₀ aryloxy such as phenoxy, naphthyloxy; aralkyloxy such as benzyloxy; alkanoate such as acetate, propanoate, butanoate, isobutyrate; aryloate such as benzoate, naphthoate; alkyl sulphonyloxy such a mesyloxy, ethane suphonyloxy; arylsulphonyloxy such as p-tolylsulfonate, p-nitrobenzenesulfonates; a substituted or unsubstituted or cyclic or acyclic amino that can form amide bond.

9. A process for preparing suvorexant (Formula I) comprising conversion of the compound of Formula IX to the compound of formula X:

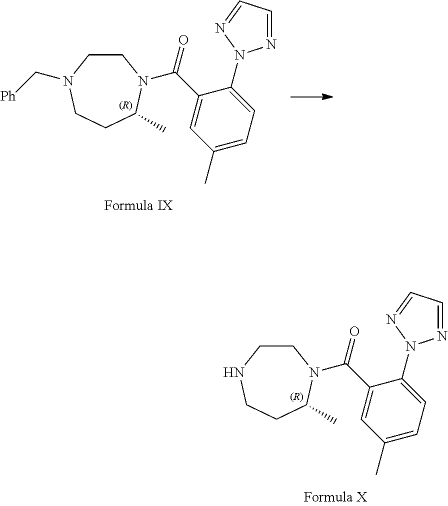

10. A process for preparing enantiomer of suvorexant (Formula Ia) comprising conversion of the compound of Formula IXa to the compound of formula Xa:

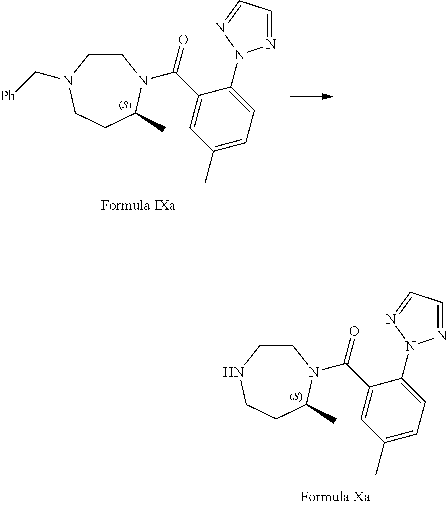

11. A process for preparing suvorexant (Formula I) comprising reaction of the compound of Formula X with the compound of formula XI:

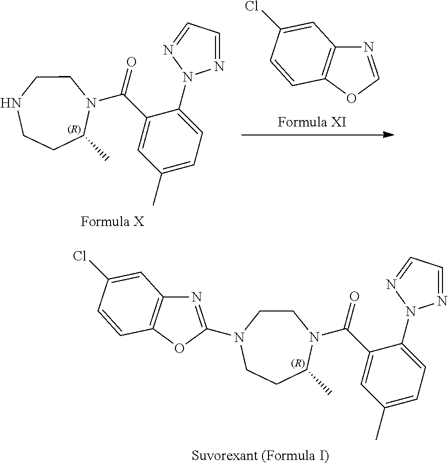

12. A process for preparing enantiomer of suvorexant (Formula Ia) comprising reaction of a compound of Formula Xa with a compound of formula XIa:

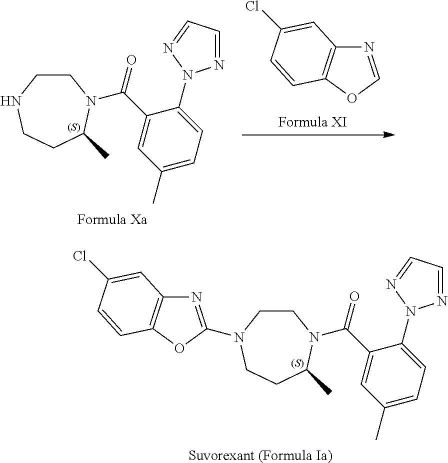

13. The process as claimed in claim 1, wherein the compound of formula II is reacted with the compound of formula XII in presence of a base to get compound of Formula III. 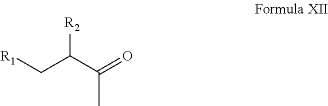
wherein R₁ includes halo (Cl, Br, I), alkyl sulphonyl, aryl sulphonyl, hydroxyl and R₂ is hydrogen; or R₁ and R₂ together form a bond; or an equivalent compound thereof.

14. The process of claim 13, wherein the compound of Formula XII includes, 4-halobutan-2-one, 3-oxobutyl methanesulfonate, 3-oxobutyl p-tolylsulfonate, 3-oxobutyl p-nitrobenzenesulfonates, pent-4-en-2-one or 4-hydroxybutan-2-one.

15. The process as claimed in claim 13, wherein the base includes, organic base such as DBU (1,8-diazabicyclo[5.4.0]undec-7-ene), DBN (1,5-Diazabicyclo[4.3.0]non-5-ene), DABCO (1,4-diaza-bicyclo[2.2.2]octane), ABCO (1-azabicyclo [2,2,2]octane), TBD (1,5,7-Triazabicyclo[4.4.0]dec-5-ene) or DMAP (4-dimethylaminopyridine), TEA (Triethylamine), DIPEA (N,N-diisopropylethylamine), DIEA (Diethylamine), N-methyl morpholine, lutidine, pyridine or collidine; hydroxides of alkali metals such as sodium hydroxide, lithium hydroxide or potassium hydroxide; carbonates of alkali metals such as sodium carbonate or potassium carbonate; bicarbonates of alkali metals such as sodium bicarbonate or potassium bicarbonate.

16. The process as claimed in claim 1, wherein conversion of the compound of Formula IV to the compound of formula VI is carried out in one pot without isolation of compound V.

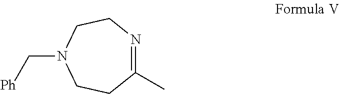

17. The process as claimed in claim 4, wherein the said conversion is carried out in presence of suitable reagent such as an acid which includes, methanesulfonic acid, ethanesulfonic acid, benzenesulfonic acid, p-toluenesulfonic acid, nitric acid, sulfuric acid, phosphoric acid, hydrochloric acid, hydrobromic acid, tartaric acid, citric acid, acetic acid, and maleic acid; or suitable reagent capable of releasing an acid in situ which includes, cyanuric halide (Formula XII), trihaloisocyanuric acid (Formula XIV), N-halosuccinimide (Formula XV), Tetrahaloglycouril (Formula XVI), 1,3-dihalo-5,5-dimethyl-hydantoin (Formula XVII), 1,3-dihalo-5,5-diphenyl-hydantoin (Formula XVIII), N-halophthalimide (Formula XIX), or N-haloacetamide (Formula XX).

18. The process as claimed in claim 4, wherein the said conversion is carried out in presence of suitable reducing agent which includes, borohydrides such as sodium borohydride, potassium borohydride, lithium borohydride, sodium cyanoborohydride, potassium cyanoborohydride, lithium cyanoborohydride, sodium triacetoxyborohydride, potassium triacetoxyborohydride, also in the presence of suitable additives such as sulfuric acid, methanesulfonic acid, acetic acid, titanium chloride, zinc chloride, cobalt (II) chloride, aluminium chloride, tin chloride, nickel chloride, phosphorus oxychloride, methanesulfonic anhydride, trifluoromethanesulfonic anhydride, pyridine, iodine, trifluoroethanol or 1,2-ethanedithiol; boranes such as borane, diborane or catechol borane, also in the form of complexes with ethers, sulfides or amines such as BH₃.SMe₂, BH₃.Et₂O, BH₃.THF, BH₃-t-butylamine, BH₃-dimethylamine or BH₃.diethylaniline; silanes such as triethylsilane, diphenylsilane or trichlorosilane, optionally in the presence of one or more Lewis acids, such as trifluoroborane, titanium chloride, aluminium chloride, zinc iodide or trifluoroacetic acid, also in form of complexes with ethers, such as boron trifluoride diethyl etherate; aluminium hydrides such as aluminium hydride (alane), LiAlH₄, iBu₂AlH, sodium bis(2-methoxyethoxy)aluminium hydride (Red-Al) or LiHAl(OCH₃)₂, optionally in the presence of one or more Lewis acids, such as trifluoroborane, titanium chloride, aluminium chloride, zinc iodide or trifluoroacetic acid.

19. The process as claimed in claim 1, wherein the conversion of the racemic compound of Formula VI to the enantiomerically enriched compound of formula VII or enantiomerically enriched compound of formula VIIa is done by resolution method.

20. The process as claimed in claim 19, wherein the said resolution includes crystallization of enantiomer mixtures; mechanical separation of enantiomers, wherein process is carried out under thermodynamic control or kinetic control; chemical separation of enantiomers, wherein the process is carried out by conversion to diastereomers under thermodynamic control or kinetic control; by intervention of diastereomeric transition states or excited states; or subjecting to optical resolution by diasteromeric salt formation followed by separation of diatereomeric salts.

21. The process as claimed in claim 1, wherein the compound of formula V is subjected to enantioselective reduction to provide enantiomerically enriched compound of formula VII or a compound of formula VIIa.

22. The process of claim 21, wherein the said enantioselective reduction is carried out by catalytic hydrogenation reaction or catalytic hydrogen transfer reaction or enzymatic reduction.

23. The process as claimed in claim 1, wherein reaction of the enantiomerically enriched compound of Formula VII or the the enantiomerically enriched compound of Formula VIIa with the compound of formula VIII to provide a compound of formula IX or a compound of formula IXa is carried out in presence of a base which includes; organic base such as DBU (1,8-diazabicyclo[5.4.0]undec-7-ene), DBN (1,5-Diazabicyclo[4.3.0]non-5-ene), DABCO (1,4-diaza-bicyclo[2.2.2]octane), ABCO (1-azabicyclo[2,2,2]octane), TBD (1,5,7-Triazabicyclo[4.4.0]dec-5-ene), DMAP (4-dimethylaminopyridine), TEA (Triethyl amine), DIPEA (N,N-diisopropylethylamine), DIEA (Diethylamine), N-methyl morpholine, lutidine, pyridine or collidine; or an inorganic base such as, hydroxides of alkali metals such as sodium hydroxide, lithium hydroxide or potassium hydroxide; carbonates of alkali metals such as sodium carbonate or potassium carbonate; or bicarbonates of alkali metals such as sodium bicarbonate or potassium bicarbonate.

24. The process as claimed in claim 1, wherein conversion of the compound of Formula IX or a compound of formula IXa to the compound of formula X or a compound of formula Xa respectively is carried out in presence of palladium based catalyst such as palladium on carbon (Pd/C); by catalytic hydrogenation reaction using hydrogen gas or hydrogen transfer reagent such as formic acid, ammonium formate or phosphoric acid.

25. The process as claimed in claim 1, wherein reaction of the compound of Formula X or a compound of formula Xa with the compound of formula XI to provide Suvorexant of formula I or its enantiomer of formula Ia is carried out in presence of a transition metal catalyst such as copper catalyst, which includes Cu(OAc)₂, CuCl₂, CuBr₂; RuCl₃; or Pd(OAc)₂.

26. The process as claimed in claim 1, wherein reaction of the compound of Formula X or a compound of formula Xa with the compound of formula XI to provide Suvorexant of formula I or its enantiomer of formula Ia is carried out in presence of in presence of an acid, which includes acetic acid, benzoic acid, 4-nitrobenzoic acid, or 4-methoxybenzoic acid; and/or in presence of oxygen or a reagent which can provide oxygen.

27. A process for preparing the compound of formula XXI: 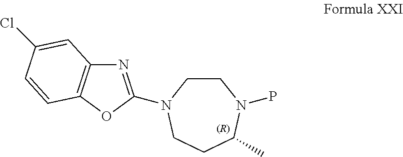
wherein P is an amino protecting group which includes, aryloxycarbonyl such as benzyloxycarbonyl (Cbz), fluorenylmethoxycarbonyl (Fmoc); alkoxycarbonyl such as methyloxycarbonyl, acetoxycarbonyl, propoxycarbonyl, tert-butyloxycarbonyl (Boc); acyl such as acetyl, propanoyl, iso-butyryl, tert-butyryl, t-butylacetyl, pivaloyl; aroyl groups such as benzoyl; silyl such as trimethylsilyl, ter-butyldimethylsilyl; sulphonyl such as methanesulphonyl, p-tolylsulphonyl; sulphenyl such as 2-nitorphenylsulfenyl; urea; urethane; nitroso; and nitro,
which comprises:
(a) protecting the compound of formula VII to provide the compound of formula XXII: 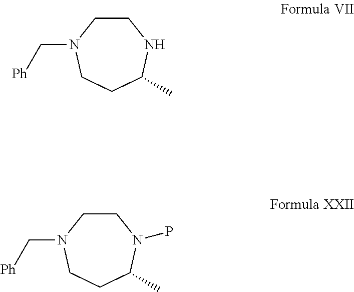
(b) converting the compound of formula XXII to obtain the compound of formula XXIII: 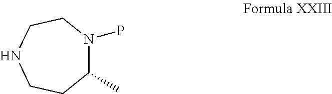
and
(c) reacting the compound of formula XXIII with the compound of formula XI: 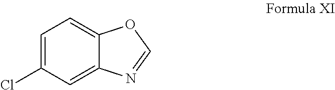
to provide a compound formula XXI.

28. The process as claimed in claim 27, wherein protection of the compound of formula VII to provide the compound of formula XXII is carried out using amino-protecting reagents such as di-tert-butyl dicarbonate, acylating reagents, sulfonylating reagents, sulfenylating reagents, urea type reagent, urethane-type reagents, nitroso derivatives, nitro derivatives, or silyl reagents.

29. The process as claimed in claim 27, wherein conversion of the compound of formula XXII to the compound of formula XXIII is carried out in presence of palladium based catalyst such as palladium on carbon (Pd/C); by catalytic hydrogenation reaction using hydrogen gas or hydrogen transfer reagent such as formic acid, ammonium formate or phosphoric acid.

30. The process as claimed in claim 27, wherein reaction of the compound of formula XXIII with the compound of formula XI to provide a compound formula XXI is carried out in presence of a transition metal catalyst such as copper catalyst, which includes Cu(OAc)₂, CuCl₂ or CuBr₂; RuCl₃; Pd(OAc)₂.

31. The process as claimed in claim 27, wherein reaction of the compound of formula XXIII with the compound of formula XI to provide a compound formula XXI is optionally carried out in presence of an acid, which includes acetic acid, benzoic acid, 4-nitrobenzoic acid, or 4-methoxybenzoic acid; and/or in presence of oxygen or a reagent which can provide oxygen.

32. A process for the preparation of a compound of formula XXV, which comprises: 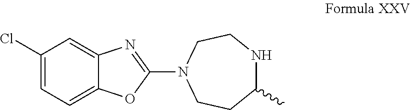 [SMILES: CC1CCN(c2nc3cc(Cl)ccc3o2)CCN1]
converting the compound of formula XXIV to the compound of formula XXV.

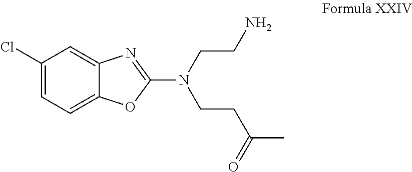 [SMILES: CC(=O)CCN(CCN)c1nc2cc(Cl)ccc2o1]

33. The process as claimed in claim 32, wherein conversion of the compound of Formula XXIV to the compound of formula XXV is carried out with or without isolation of compound XXIVa.

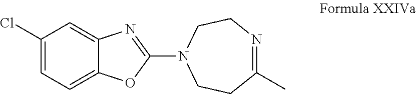 [SMILES: CC1=NCCN(c2nc3cc(Cl)ccc3o2)CC1]

34. The process as claimed in claim 32, wherein conversion is carried out in presence suitable reagent such as an acid or a reagent capable of releasing an acid in situ.

35. The process as claimed in claim 34, wherein the suitable acid includes, methanesulfonic acid, ethanesulfonic acid, benzenesulfonic acid, p-toluenesulfonic acid, nitric acid, sulfuric acid, phosphoric acid, hydrochloric acid, hydrobromic acid, tartaric acid, citric acid, acetic acid, and maleic acid.

36. The process as claimed in claim 34, wherein the suitable reagent capable of releasing an acid in situ includes, cyanuric halide (Formula XII), trihaloisocyanuric acid (Formula XIV), N-halosuccinimide (Formula XV), Tetrahaloglycouril (Formula XVI), 1,3-dihalo-5,5-dimethyl-hydantoin (Formula XVII), 1,3-dihalo-5,5-diphenyl-hydantoin (Formula XVIII), N-halophthalimide (Formula XIX), or N-haloacetamide (Formula XX).

37. The process as claimed in claim 32, wherein conversion is carried out in presence suitable reducing agent which includes, borohydrides such as sodium borohydride, potassium borohydride, lithium borohydride, sodium cyanoborohydride, potassium cyanoborohydride, lithium cyanoborohydride, sodium triacetoxyborohydride, potassium triacetoxyborohydride, also in the presence of suitable additives such as sulfuric acid, methanesulfonic acid, acetic acid, titanium chloride, zinc chloride, cobalt (II) chloride, aluminium chloride, tin chloride, nickel chloride, phosphorus oxychloride, methanesulfonic anhydride, trifluoromethanesulfonic anhydride, pyridine, iodine, trifluoroethanol or 1,2-ethanedithiol; boranes such as borane, diborane or catechol borane, also in the form of complexes with ethers, sulfides or amines such as BH₃.SMe₂, BH₃.Et₂O, BH₃.THF, BH₃-t-butylamine, BH₃-dimethylamine or BH₃.diethylaniline; silanes such as triethylsilane, diphenylsilane or trichlorosilane, optionally in the presence of one or more Lewis acids, such as trifluoroborane, titanium chloride, aluminium chloride, zinc iodide or trifluoroacetic acid, also in form of complexes with ethers, such as boron trifluoride diethyl etherate; aluminium hydrides such as aluminium hydride (alane), LiAlH₄, iBu₂AlH, sodium bis(2-methoxyethoxy)aluminium hydride (Red-Al) or LiHAl(OCH₃)₂, optionally in the presence of one or more Lewis acids, such as trifluoroborane, titanium chloride, aluminium chloride, zinc iodide or trifluoroacetic acid.
